(19) United States
(12) Patent Application Publication
(10) Pub. No.: US 2003/0083546 A1
(43) Pub. Date: May 1, 2003

(54) Device

(76) Inventors: John Butler, Blackrock (IE); Frank Bonadio, Bray (IE); Duncan Bell, Chilton Moor (GB); Lisa Woods, Ardee (IE); Trevor Vaugh, Birr (IE)

Correspondence Address: Finnegan, Henderson, Farabow,, Garrett & Dunner, L.L.P., 1300 I Street, N.W., Washington, DC, 20005-3315, US

(21) Appl. No.: 10/271,764

(22) Filed: Oct. 17, 2002

(30) Foreign Application Priority Data
Oct. 18, 2001 (IE) 2001/0915
Feb. 20, 2002 (IE) 2002/0128

(51) Int. Cl. A61B 1/00

(52) U.S. Cl. 600/114

(57) ABSTRACT

A device 1 to aid advancement of a colonoscope 2 through the colon 3 of a patient comprises an elongate body 4 defining a colonoscope lumen 7. The body 4 provides a fluid tight seal to the colonoscope 2. The body 4 has an anchor to releasably anchor the interior wall of a colon 3 to the device to enable a colonoscope 2 to be more easily advanced. The anchor may be provided by suction apertures 8 through which a suction is applied to draw the colon into anchored engagement with the body 4. The anchor may be provided by a balloon and/or foam based anchoring systems may be provided.

### INTRODUCTION

This invention relates to a device to aid advancement of a colonoscope through a colon. In particular, the invention relates to a device to aid advancement of a colonoscope through a sigmoid colon

Conventional colonoscopy procedures involve advancing a colonoscope through the floppy sigmoid colon to the proximal end of the descending colon.

However, advancing a colonoscope through the sigmoid colon generally causes loops to form in the floppy sigmoid colon, and stretches the mesentery to which the sigmoid colon is attached. This results in considerable pain and discomfort for the patient.

In addition, it is often difficult to advance a colonoscope through a colon due to the presence of an obstruction to advancement of the colonoscope through the colon, such as a tight bend in the sigmoid colon, or a protruding piece of the colon wall, or a polyp on the colon wall.

This invention provides a device to aid advancement of a colonoscope through a colon which is aimed at overcoming at least some of these problems.

### STATEMENTS OF INVENTION

According to the invention there is provided a device to aid advancement of a colonoscope through a colon, the device comprising:

an elongate body extending between a proximal end for location externally of a colon and a distal end for insertion into a colon;

the body defining a colonoscope lumen therethrough, and

an anchor to releasably anchor the interior wall of a colon to the device.

In one embodiment of the invention the anchor comprises at least one suction aperture in the body to draw the interior wall of a colon into an anchored engagement with the body by suction. The anchor may comprise a plurality of suction apertures. Preferably the apertures are evenly spaced around the body. Ideally the apertures are evenly spaced along the body.

In one case the anchor comprises at least one suction lumen in fluid communication with the suction aperture. Most preferably the suction lumen extends proximally from the suction aperture to the proximal end of the body. Ideally the suction lumen is provided by the colonoscope lumen. Desirably the anchor comprises a source of negative gauge pressure in fluid communication with the suction aperture.

In another embodiment of the invention the anchor comprises at least one member which is moveable outwardly into an anchored engagement with the interior wall of a colon. The member may be expandable into an anchored engagement with the interior wall of a colon. Preferably the member is inflatable into an anchored engagement with the interior wall of a colon. Most preferably the inflatable member comprises a balloon.

In one case the anchor comprises at least one inflation lumen in fluid communication with the inflatable member. Most preferably the inflation lumen extends proximally from the inflatable member to the proximal end of the body. Ideally the inflation lumen is provided by the colonoscope lumen. Desirably the anchor comprises an inflation fluid source in fluid communication with the inflatable member.

In one embodiment of the invention the anchor comprises an absorber. The absorber may be mounted to the elongate body.

In one case the absorber is biased inwardly. In an alternative case the absorber is biased outwardly.

The absorber may be mounted to or comprise the movable member.

In one embodiment the absorber comprises at least one pad on the device.

In another embodiment the absorber comprises at least one strip extending along the device. The strip may extend substantially longitudinally. The strip may extend substantially in a spiral or part thereof.

In another case the absorber comprises at least one hoop extending around the device. It is believed that such hoop(s) enhance the anchoring of the interior wall of a colon to the device due to the rippled nature of the colon wall.

The absorber may surround the anchor. Ideally the absorber has a plurality of openings therein. Alternatively the absorber may be a continuous enclosure around the anchor.

Preferably the absorber is of a porous material. The porous material may be of an open cell construction, such as a polymeric material.

In a preferred embodiment the device comprises a sleeve to separate the absorber from the interior wall of a colon during insertion and removal of the device. Ideally the sleeve comprises a movable sheath.

It is believed that the absorber enhances the anchoring of the interior wall of a colon to the device.

It is believed that better traction between the device of the invention and the interior wall of a colon is achieved by increasing the frictional forces acting on the surface(s) of contact.

The sleeve enables the device to be inserted into and removed from a colon with less frictional resistance.

The anchor is preferably provided adjacent the distal end of the body.

In a preferred embodiment of the invention the body comprises at least one seal for sealing between the body and a colonoscope within the colonoscope lumen. Preferably the seal is a fluid-tight seal. Desirably the seal is provided at an end of the body. Most preferably the body comprises a distal seal at the distal end of the body and a proximal seal at the proximal end of the body.

In another aspect, the invention provides a colonoscopy apparatus, the apparatus comprising:

a device according to the invention; and

a colonoscope defining a proximal end for location externally of a colon and a distal end for insertion into a colon.

In one embodiment the colonoscope comprises an insufflation means at the distal end of the colonoscope to insufflate a colon distally of the colonoscope to clear a passage for advancement of the colonoscope through the colon. Preferably the insufflation means comprises at least one insufflation aperture in the colonoscope. Ideally the insufflation means comprises at least one insufflation lumen in fluid communication with the insufflation aperture. Most preferably the insufflation lumen extends proximally from the insufflation aperture to the proximal end of the colonoscope. Desirably the insufflation means comprises an insufflation fluid source in fluid communication with the insufflation aperture.

In a preferred embodiment the apparatus comprises a sealing means to create a fluid-tight seal to the interior wall of a colon proximally of the distal end of the colonoscope during insufflation. Ideally the sealing means is provided by the anchor.

According to a further aspect of the invention, there is provided a method of advancing a colonoscope through a colon, the method comprising the steps of:

providing a colonoscope, the colonoscope defining a proximal end and a distal end;

providing an anchoring device, the anchoring device defining a proximal end and a distal end;

inserting the distal end of the colonoscope into a colon;

inserting the distal end of the anchoring device into the colon;

anchoring the interior wall of the colon to the anchoring device;

advancing the colonoscope through at least part of the colon; and

releasing the anchor.

In one embodiment the method comprises the step of moving the anchoring device proximally to at least partially straighten the colon after anchoring the interior wall of the colon to the anchoring device. The method may comprise the step of moving the colonoscope proximally during the step of moving the anchoring device proximally. The method may alternatively comprise the step of maintaining the position of the colonoscope fixed during the step of moving the anchoring device proximally.

In a preferred embodiment the method comprises the step of insufflating the colon distally of the colonoscope to clear a passage for advancement of the colonoscope through the colon. Ideally the method comprises the step of creating a fluid-tight seal to the interior wall of the colon proximally of the distal end of the colonoscope before insufflating the colon. Desirably the seal to the interior wall of the colon is created by the step of anchoring the interior wall of the colon to the anchoring device.

In another embodiment the interior wall of the colon is anchored to the anchoring device by applying suction to draw the interior wall of the colon into an anchored engagement with the anchoring device. Preferably the anchor is released by releasing the suction.

In a further embodiment the interior wall of the colon is anchored to the anchoring device by moving at least part of the anchoring device outwardly into an anchored engagement with the interior wall of the colon. Ideally the part of the anchoring device is moved outwardly by inflation. Most preferably the anchor is released by moving the part of the anchoring device inwardly.

In a preferred embodiment the method comprises the step of absorbing fluid from the colon using the anchoring device.

In one case the method comprises the step of advancing the colonoscope through the colon until the distal end of the colonoscope encounters an obstruction to further advancement of the colonoscope through the colon before anchoring the interior wall of the colon to the anchoring device. The colonoscope may be advanced through the colon until the distal end of the colonoscope is at the proximal end of the sigmoid colon before anchoring the interior wall of the colon to the anchoring device.

Preferably at least some of the steps are repeated at least once to advance the colonoscope through the colon in an incremental manner. Most preferably the location of anchoring of the interior wall of the colon to the anchoring device progresses successively distally as the steps are repeated.

The anchoring device may be passed over the colonoscope. Ideally the anchoring device is passed over the colonoscope in a sealed manner.

According to a further aspect of the invention, there is provided a method of advancing a colonoscope through a sigmoid colon, the method comprising the steps of:

providing a colonoscope, the colonoscope defining a proximal end and a distal end;

providing an anchoring device, the anchoring device defining a proximal end and a distal end;

inserting the distal end of the colonoscope into a sigmoid colon; inserting the distal end of the anchoring device into the sigmoid colon;

anchoring the interior wall of the sigmoid colon to the anchoring device;

advancing the colonoscope through at least part of the sigmoid colon; and

releasing the anchor.

In one case the method comprises the step of moving the anchoring device proximally to at least partially straighten the sigmoid colon after anchoring the interior wall of the sigmoid colon to the anchoring device.

In another case, the invention provides a method of straightening a sigmoid colon. Preferably the method comprises the steps of:

advancing the anchoring device until the distal end of the anchoring device is at the proximal end of the descending colon to splint the straightened sigmoid colon; and

advancing the colonoscope into the descending colon.

The device of the invention may be used to anchor the interior wall of a colon to the device. By holding the device in a fixed position, the colon is also held in a fixed position. It is thus easier to advance a colonoscope through the fixed colon, and less pain/discomfort will be caused to the patient. In the case of using the device to aid advancement of a colonoscope through a sigmoid colon, the device prevents loops from forming in the sigmoid colon, and stretching of the mesentery to which the sigmoid colon is attached is also minimised.

The device facilitates the advancement of the colonoscope through the colon in an incremental manner with relatively small advancement steps of the colonoscope and the device through the colon between each anchoring manoeuvre.

The device of the invention may also be used to collapse the sigmoid colon of a patient to a reduced, straightened configuration substantially without causing stretching of the colon and the mesentery to which the colon is attached, and causing the resultant pain and discomfort to the patient.

It is not necessary to advance the colonoscope all the way through the sigmoid colon to the proximal end of the descending colon before beginning the reduction of the sigmoid colon using the device of the invention.

The device of the invention may be used to aid advancement of a colonoscope through a colon while insufflating the colon distally of the colonoscope. The anchoring of the interior wall of the colon to the device proximally of the insufflation aperture prevents the insufflation fluid from leaking proximally and in this manner an effective, higher pressure insufflation of the colon is achieved.

### BRIEF DESCRIPTION OF THE DRAWINGS

The invention will be more clearly understood from the following description of some embodiments thereof, given by way of example only, with reference to the accompanying drawings, in which:

FIG. 1 is a longitudinal, side view of a device according to the invention passing over a colonoscope in a colon;

FIG. 2 is a longitudinal, side view of the interior wall of the colon anchored to the device of FIG. 1;

FIG. 3(a) is a schematic view illustrating partial insertion of a colonoscope into a colon of a patient;

FIG. 3(b) is a schematic view illustrating partial insertion of the device of FIGS. 1 and 2 into the colon over the colonoscope;

FIG. 3(c) is a schematic view illustrating anchoring of the interior wall of the colon to the device by suction;

FIG. 4 is a schematic view illustrating drawing of the colon proximally to partially straighten the sigmoid colon;

FIG. 5 is a schematic view illustrating advancing the colonoscope through the colon;

FIG. 6 is a schematic view illustrating releasing the anchor of the interior wall of the colon to the device;

FIG. 7 is a schematic view illustrating advancing the device through the colon over the colonoscope;

FIG. 8 is a schematic view illustrating anchoring of the interior wall of the colon to the device by suction;

FIG. 9 is a schematic view illustrating drawing of the colon proximally to partially straighten the sigmoid colon;

FIGS. 10 to 18 are schematic views, similar to FIGS. 3(a) to 9, illustrating straightening of the sigmoid colon using another device according to the invention;

FIG. 19 is a perspective view of another device according to the invention in an uninflated configuration passing over the colonoscope;

FIG. 20 is a perspective view of the device of FIG. 19 in an inflated configuration passing over the colonoscope;

FIGS. 21 and 22 are views similar to FIGS. 19 and 20 of another device according to the invention passing over the colonoscope;

FIG. 23 is a perspective view of another device according to the invention in an uninflated configuration passing over the colonoscope;

FIG. 24 is a perspective view of the device of FIG. 23 in an inflated configuration passing over the colonoscope;

FIGS. 25 and 26 are perspective views in the opposite direction of the device of FIGS. 23 and 24 passing over the colonoscope;

FIGS. 27 and 28 are partially cross-sectional, side views of the device of FIGS. 23 and 24 passing over the colonoscope;

FIGS. 29 to 34 are views similar to FIGS. 19 and 20 of other devices according to the invention passing over the colonoscope;

FIG. 35 is a perspective view of another device according to the invention in an uninflated configuration passing over the colonoscope;

FIG. 36 is a partially cut-away, perspective view of the device of FIG. 35 in the uninflated configuration passing over the colonoscope;

FIG. 37 is a partially cut-away, perspective view of the device of FIG. 35 in an inflated configuration passing over the colonoscope;

FIG. 38 is a schematic view illustrating partial insertion of a colonoscope into a colon of a patient;

FIG. 39 is a schematic view illustrating partial insertion of the device of FIGS. 1 and 2 into the colon over the colonoscope;

FIG. 40 is a schematic view illustrating insufflating the colon after anchoring the interior wall of the colon to the device by suction;

FIG. 41 is a schematic view illustrating advancing the colonoscope through the colon;

FIG. 42 is a schematic view illustrating advancing the device through the colon over the colonoscope;

FIG. 43 is a schematic view illustrating insufflating the colon after anchoring the interior wall of the colon to the device by suction;

FIG. 44 is a schematic view illustrating releasing the anchor of the interior wall of the colon to the device after advancing the colonoscope through the colon; and

FIGS. 45 to 49 are schematic views, similar to FIGS. 38 to 42, illustrating advancing the colonoscope through the colon using the device of FIGS. 10 to 18.

### DETAILED DESCRIPTION

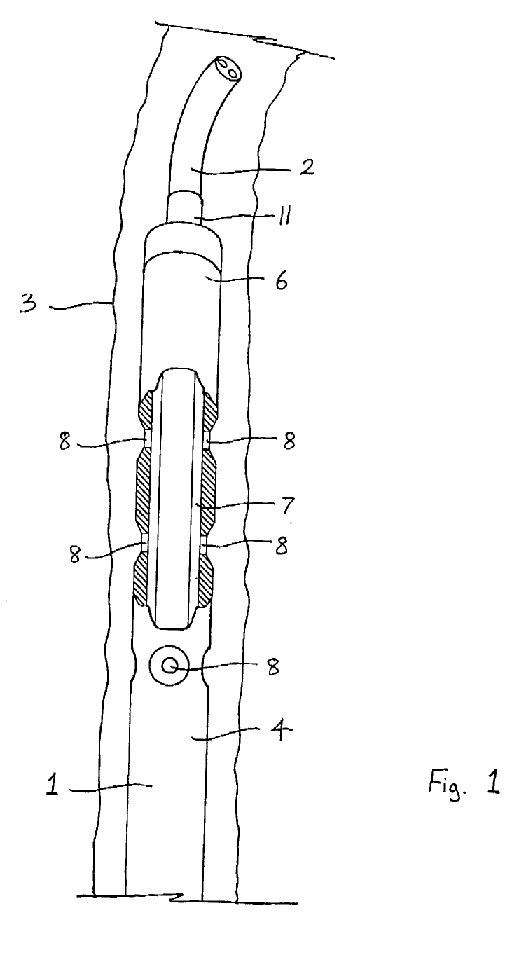

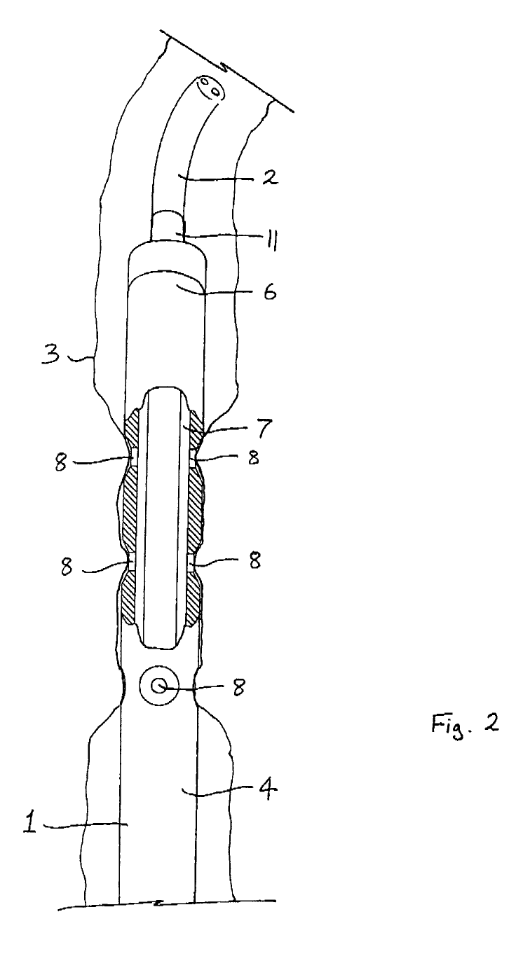

Referring to the drawings, and initially to FIGS. 1 and 2 thereof, there is illustrated a device 1 according to the invention for use to aid advancement of a colonoscope 2 through a colon 3 of a patient.

The device 1 comprises an elongate body 4 which extends between a proximal end 5 for location externally of the colon 3 and a distal end 6 for insertion into the colon 3. The body 4 defines a colonoscope lumen 7 therethrough for passing the body 4 over the colonoscope 2, and the body 4 comprises a fluid-tight distal seal 11 between the body 4 and the colonoscope 2 at the distal end 6 of the body 4, and a fluid-tight proximal seal 12 between the body 4 and the colonoscope 2 at the proximal end 5 of the body 4.

The seals 11, 12 prevent material such as faeces, bodily fluids, or parts of the colon wall from entering the colonoscope lumen 7 between the colonoscope 2 and the body 4, and possibly leaking proximally through the lumen 7 and out of the colon 3 during the colonoscopy procedure.

The distal seal 11 also prevents parts of the interior wall of the colon 3 from becoming snagged or caught on the distal end 6 of the body 4, or trapped in the colonoscope lumen 7, or in any other way damaged, as the body 4 is advanced through the colon 3.

The body 4 comprises an anchor to releasably anchor the interior wall of the colon 3 to the device 1. By anchoring the interior wall of the colon 3 to the device 1, and holding the device 1 in position, this enables the colonoscope 2 to be more easily advanced through the colon 3.

In this case, the anchor is provided by a plurality of suction apertures 8 in the body 4 adjacent the distal end 6 of the body 4. A suction lumen, which is in fluid communication with the suction apertures 8, extends proximally from the suction apertures 8 to the proximal end 5 of the body 4, where the suction lumen is connected in fluid communication with a source of negative gauge pressure, for example via a tubing 9.

By applying suction at the suction apertures 8, the interior wall of the colon 3 may be drawn into an anchored engagement with the body 4, as illustrated in FIG. 2.

The apertures 8 have a cylindrical cross-section with a tapered lead-in, as illustrated in FIGS. 1 and 2. Alternatively, the apertures 8 may have a cylindrical cross-section, or a rounded cross-section, or any suitable combination of these cross-sections.

The apertures 8 are preferably spaced evenly around the body 4 and along the body 4, as illustrated in FIGS. 1 and 2, for an even anchored engagement of the body 4 with the interior wall of the colon 3.

By maximising the number of suction apertures 8, the required suction pressure at each aperture 8 may be minimised, and thus the possibility of damaging the interior wall of the colon 3 is minimised.

The suction lumen is preferably provided by the colonoscope lumen 7. In this case, the seals 11, 12 ensure no fluid leakage occurs at the seals 11, 12 so that an effective and secure suction is applied at the apertures 8.

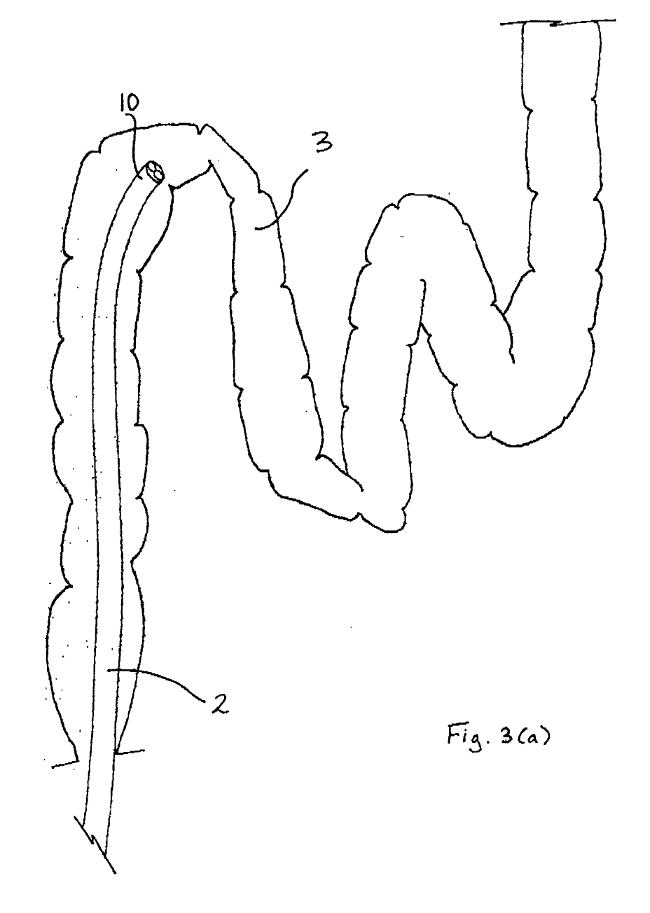

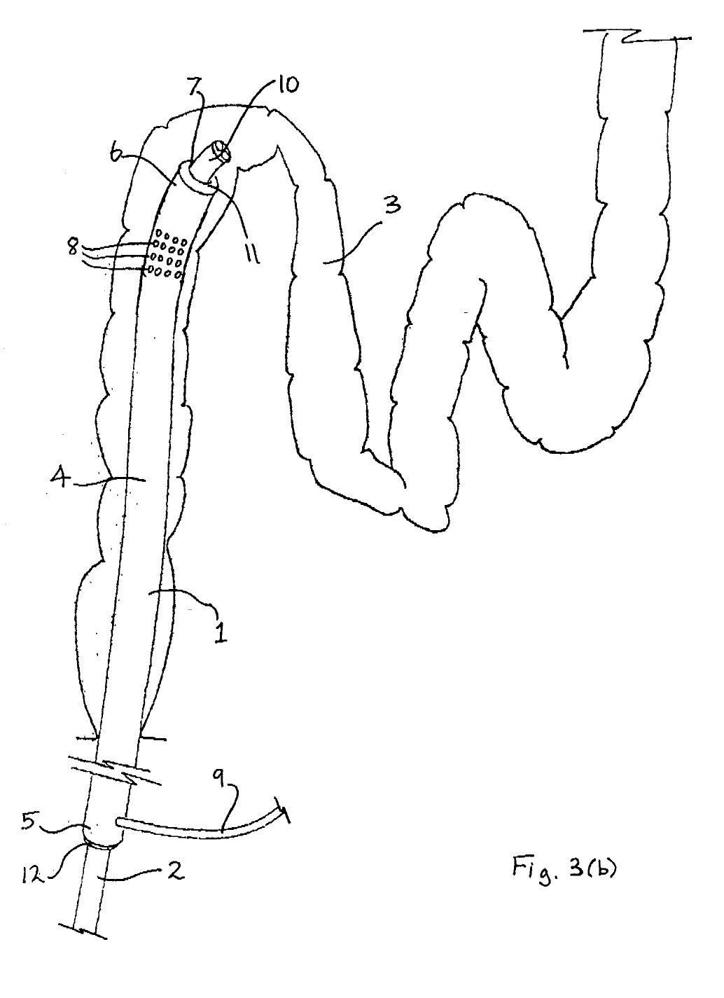

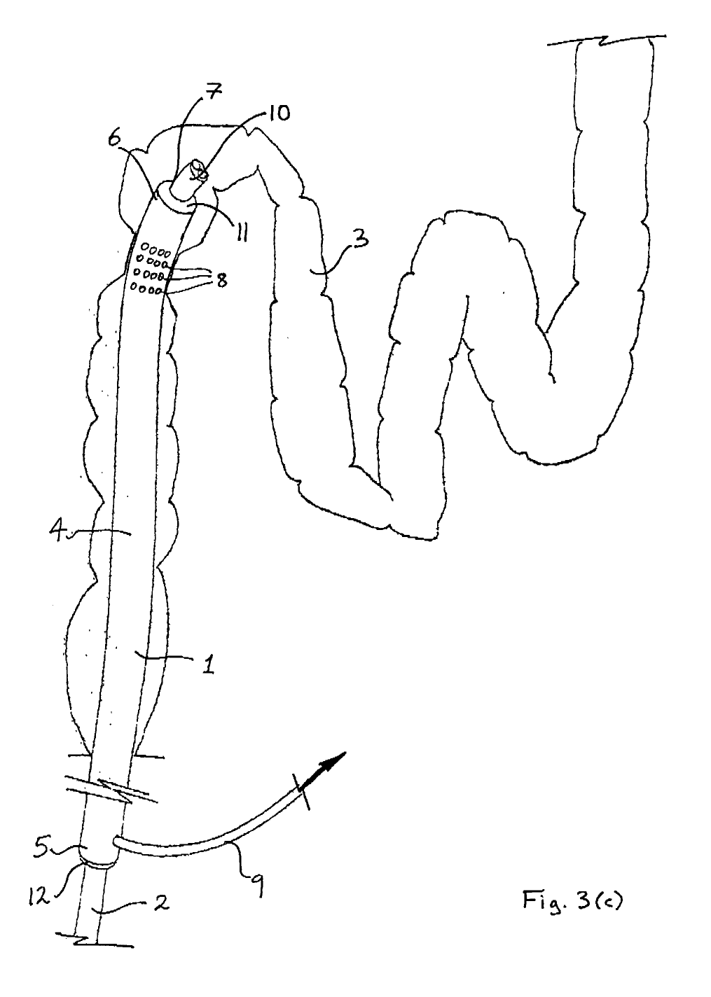

In use, a distal end 10 of the colonoscope 2 is inserted into the colon 3, and the colonoscope 2 is advanced through the colon 3 until the distal end 10 of the colonoscope 2 has encountered an obstruction to further advancement through the colon 3, for example when the distal end 10 of the colonoscope 2 has reached the proximal end of the sigmoid colon (FIG. 3(a)).

The distal end 6 of the body 4 is inserted into the colon 3 and advanced over the colonoscope 2 until the distal end 6 of the body 4 is immediately proximally of the distal end 10 of the colonoscope 2 (FIG. 3(b)). Suction is then applied at the suction apertures 8 to anchor the interior wall of the colon 3 to the device 1 by drawing the interior wall of the colon into an anchored engagement with the body 4 (FIG. 3(c)).

By holding the device 1 in position, the anchored colon 3 will also be held in position. The colonoscope 2 may then be advanced more easily through the colon 3.

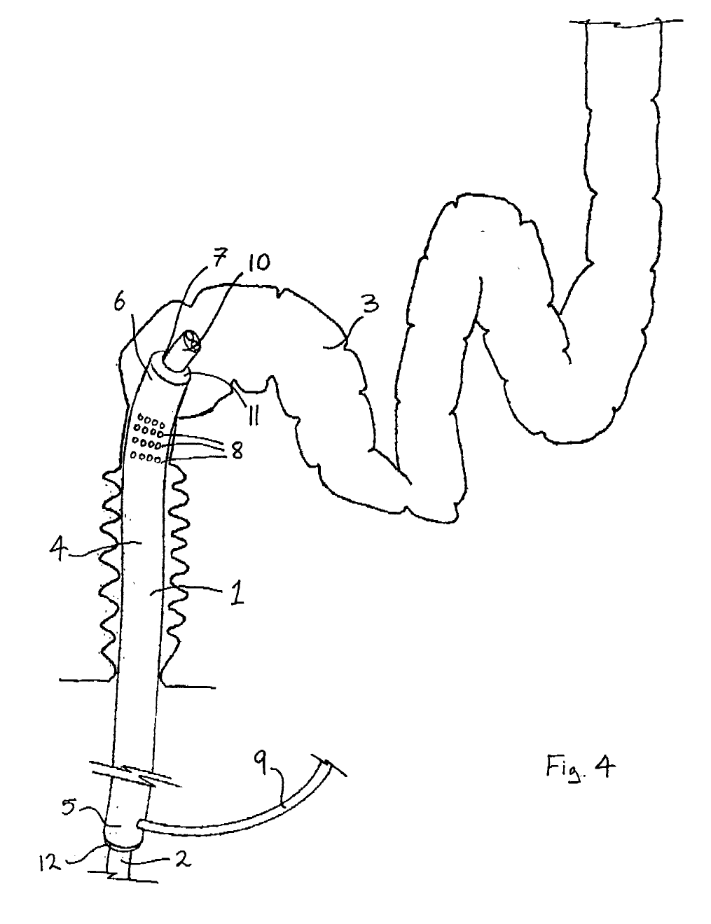

In this case, the method also includes the step of moving the body 4 proximally, to draw the anchored colon 3 proximally to partially straighten the sigmoid colon (FIG. 4).

In this case, the colonoscope 2 is retracted with the body 4 during straightening of the sigmoid colon. However, it will be appreciated that the colonoscope 2 may alternatively be held in place during movement of the body 4 only proximally, to straighten the sigmoid colon 3.

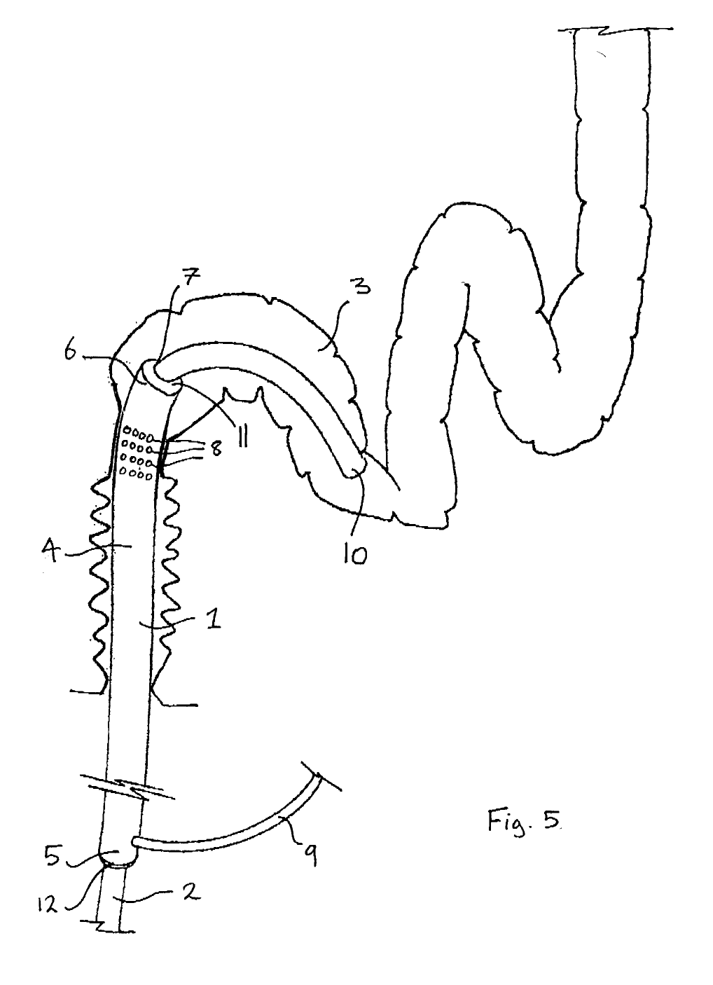

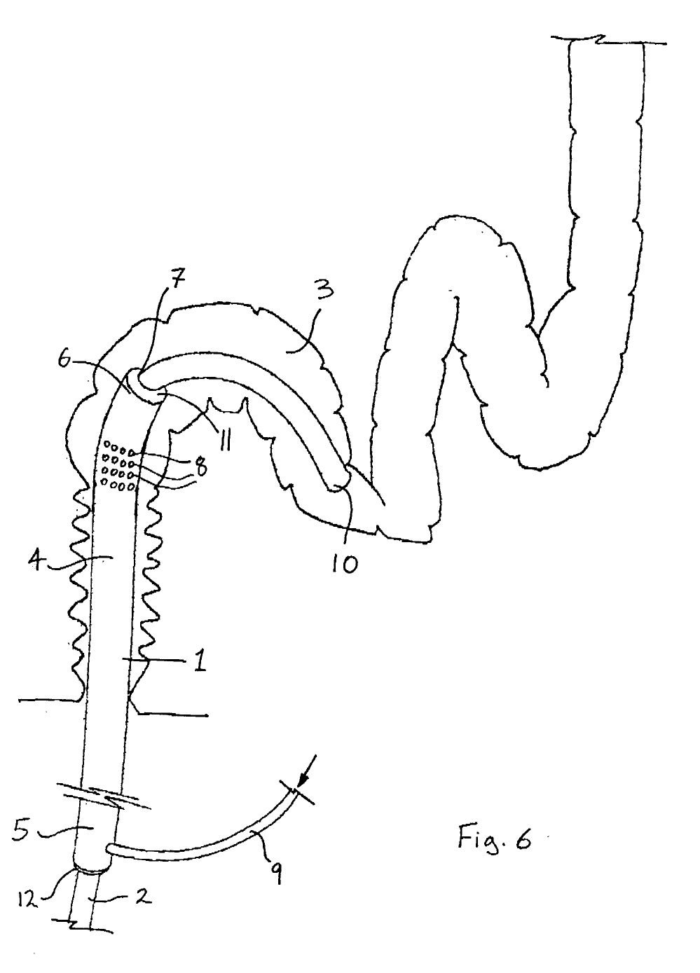

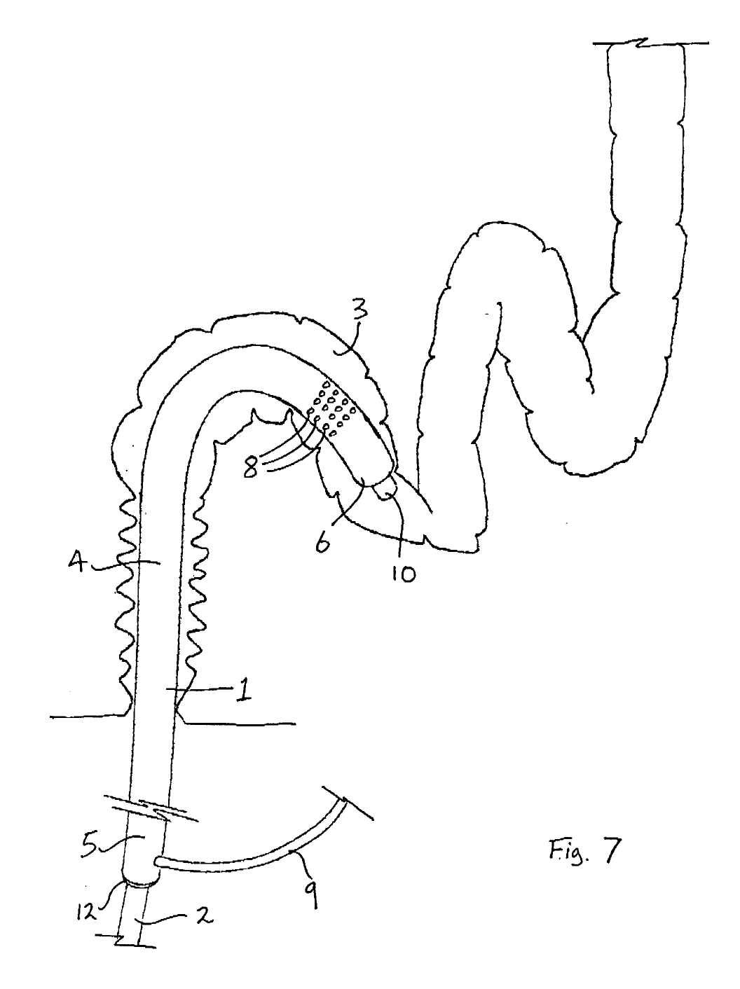

The colonoscope 2 is then advanced further distally through part of the now partially straightened sigmoid colon (FIG. 5), and the suction applied at the suction apertures 8 is released to release the anchor of the interior wall of the colon 3 to the body 4 (FIG. 6). The device 1 is thus free to be advanced distally through the colon 3 over the colonoscope 2 until the distal end 6 of the body 4 is immediately proximally of the distal end 10 of the colonoscope 2 (FIG. 7).

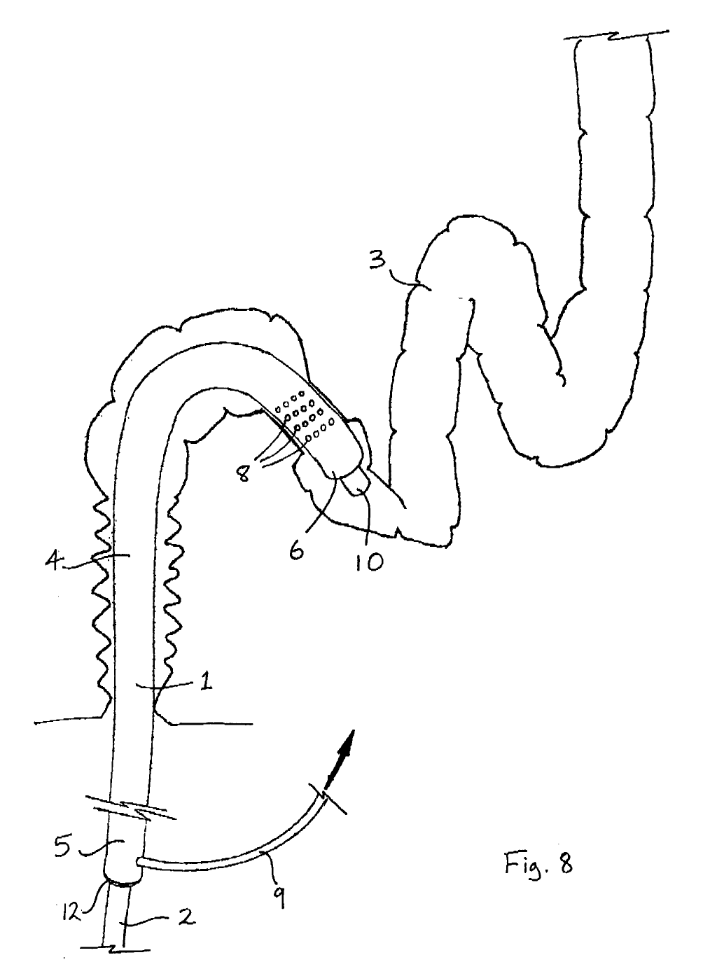

The interior wall of the colon 3 is gripped by applying suction at the suction apertures 8 (FIG. 8), and the colonoscope 2 and body 4 are moved proximally together to draw the colon 3 proximally to further straighten the sigmoid colon (FIG. 9).

As is clear from the drawings, and FIG. 8 in particular, the location of anchoring of the interior wall of the colon 3 to the device 1 has progressed distally from the initial anchoring location illustrated in FIG. 3 to the next anchoring location illustrated in FIG. 8.

The series of steps described previously with reference to FIGS. 3(a) to 9 may thus be repeated to advance the colonoscope 2 through the colon 3 in an incremental manner, and possibly additionally to straighten the sigmoid colon in an incremental manner, each time progressing the location of anchoring of the interior wall of the colon 3 to the device 1 successively distally.

When the device 1 is used to straighten a sigmoid colon the device 1 may be advanced until the distal end 6 of the body 4 is at the proximal end of the descending colon. The device 1 may thus be used as an overtube to splint the straightened sigmoid colon. The colonoscope 2 may be advanced into the descending colon and further on into the transverse colon and into the ascending colon. The device 1 prevents loops from reforming in the sigmoid colon as the colonoscope 2 advances through the colon 3.

It will be appreciated by those skilled in the art that the device 1 of the invention may be used with a colonoscope 2 to reduce a sigmoid colon to a straightened configuration in an incremental manner by repeating the steps, described previously with reference to FIGS. 5 to 9, a sufficient number of times until the sigmoid colon is straightened. The number of times the steps need to be performed depends on the physiological characteristics of the patient on whom the colonoscopy is being performed. In certain cases, it may only require one reduction to straighten the sigmoid colon sufficiently for a smooth, pain-free advancement of the colonoscope 2 through the sigmoid colon to the proximal end of the descending colon.

The first location of anchoring of the interior wall of the colon 3 to the device 1 may be proximally of the sigmoid colon depending on the physiological characteristics of the patient, and may even be in the region of the rectum.

It is believed that anchoring the interior wall of a colon 3 by means of suction results in a secure anchor of the colon wall to the device 1 without causing discomfort to the patient.

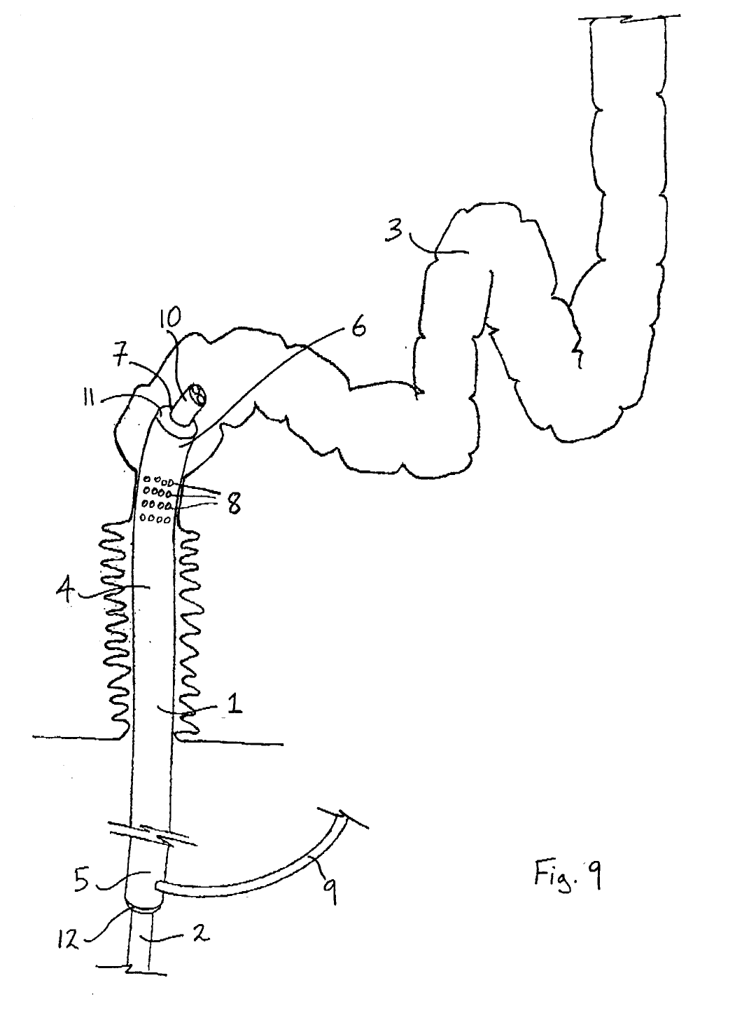

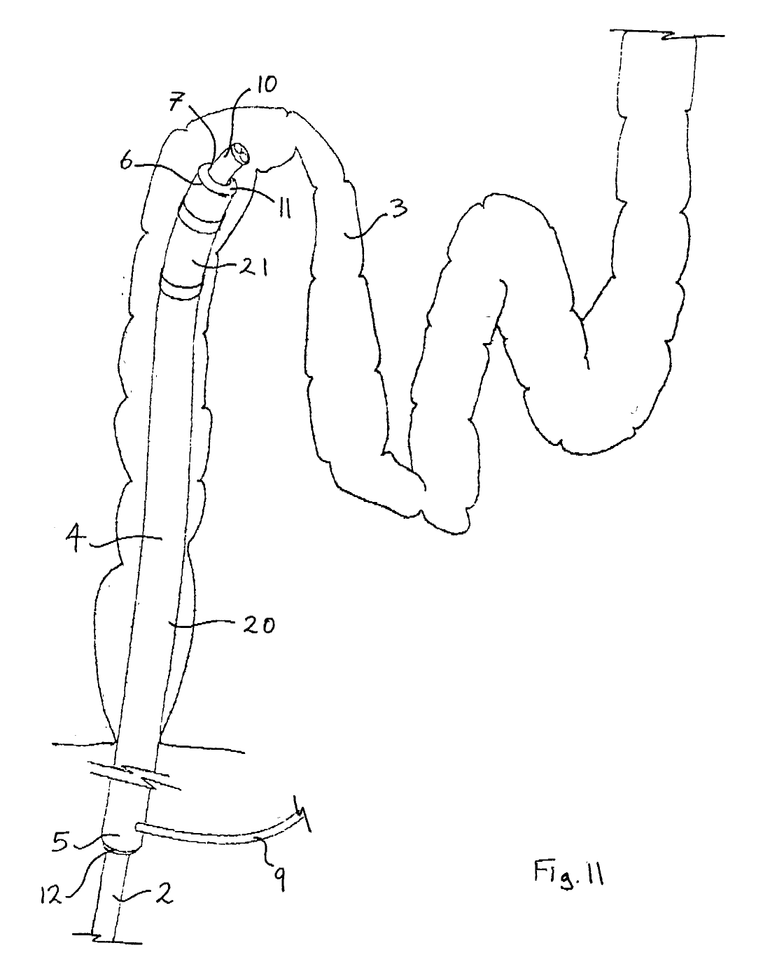

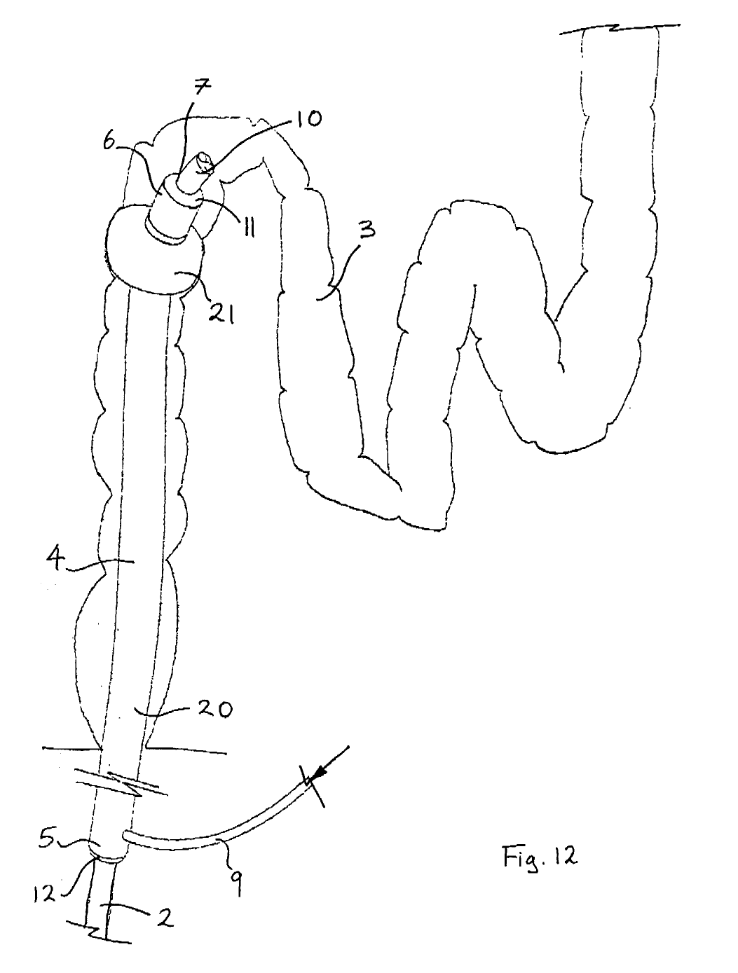

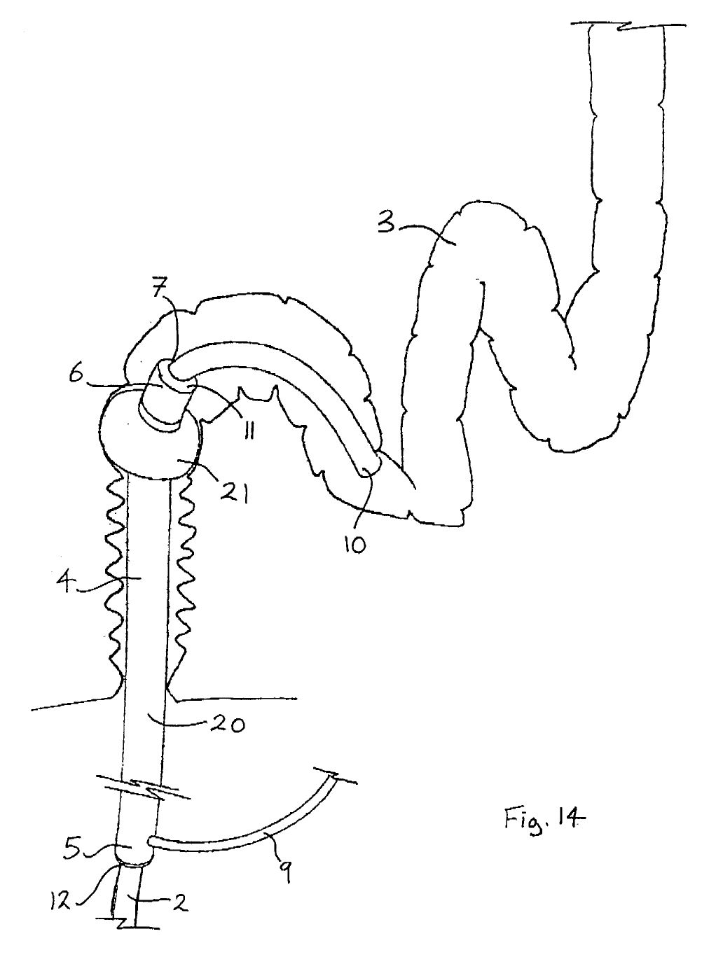

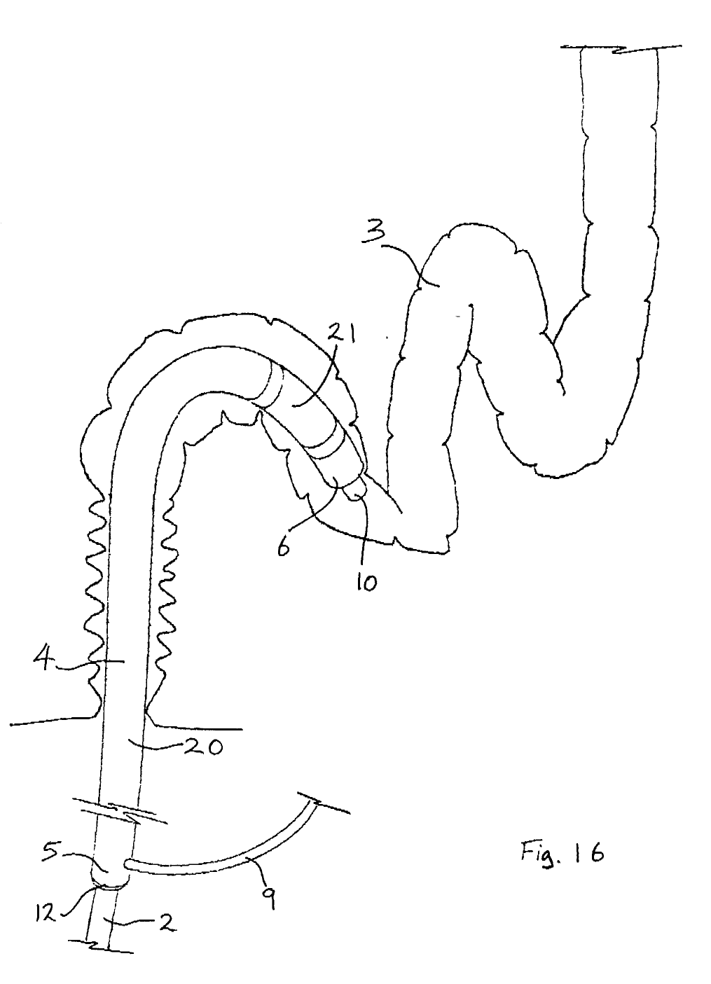

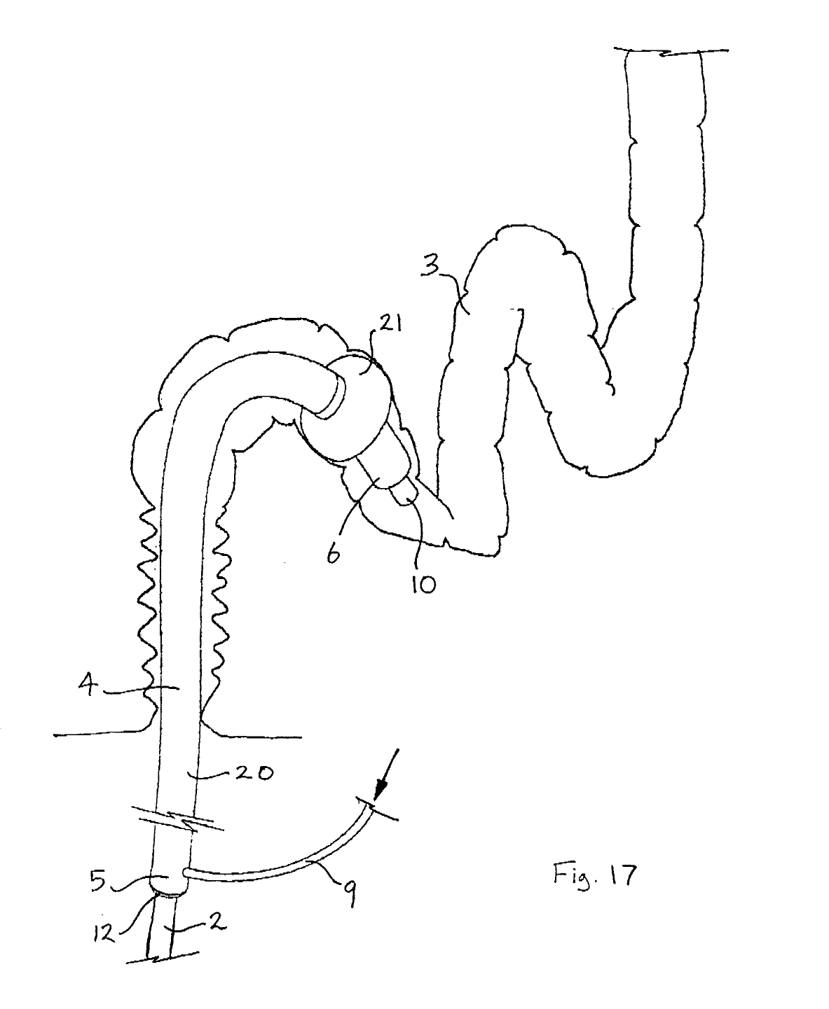

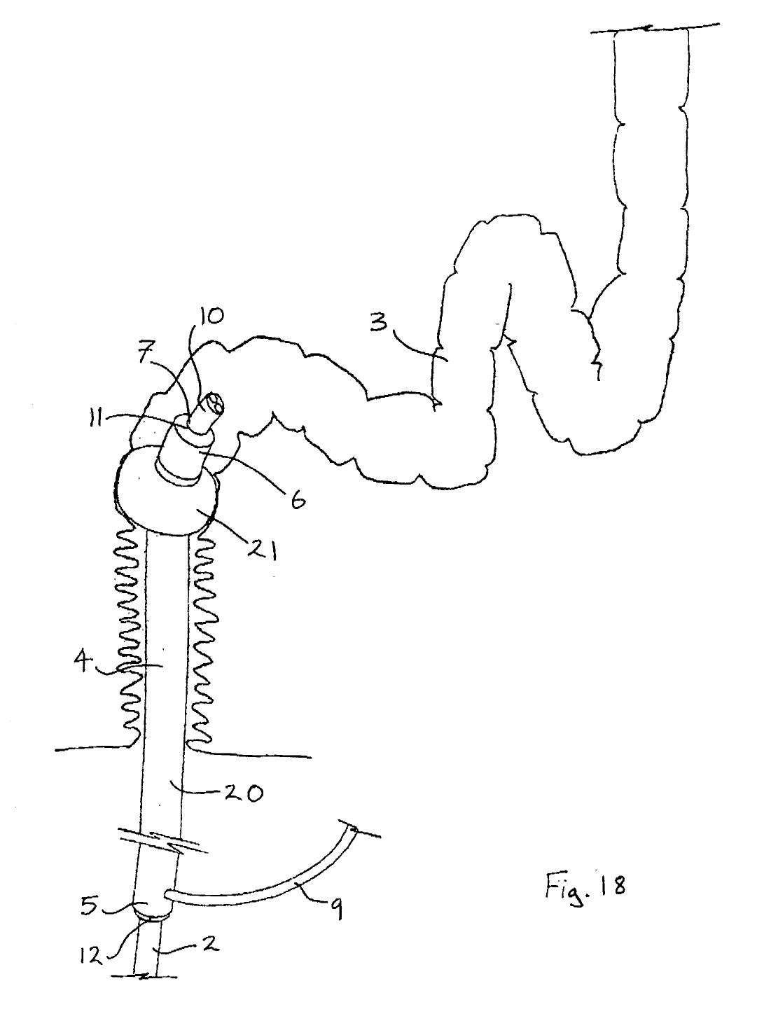

Referring to FIGS. 10 to 18, there is illustrated another device 20 according to the invention, which is similar to the device 1 of FIGS. 1 to 9, and similar elements in FIGS. 10 to 18 are assigned the same reference numerals.

The anchor is provided by at least one outwardly movable member. The member may be expandable outwardly, and is preferably inflatable into an anchored engagement with the interior wall of the colon 3. In this case, the anchor comprises a balloon 21 annularly mounted adjacent the distal end 6 of the body 4, as illustrated in FIG. 11.

The balloon 21 may be of silicone, or of an elasticated film material.

An inflation lumen, which is in fluid communication with the balloon 21, extends proximally from the balloon 21 to the proximal end 5 of the body 4, where the lumen is connected in fluid communication with an inflation fluid source, for example via the tubing 9.

The inflation lumen is preferably provided by the colonoscope lumen 7.

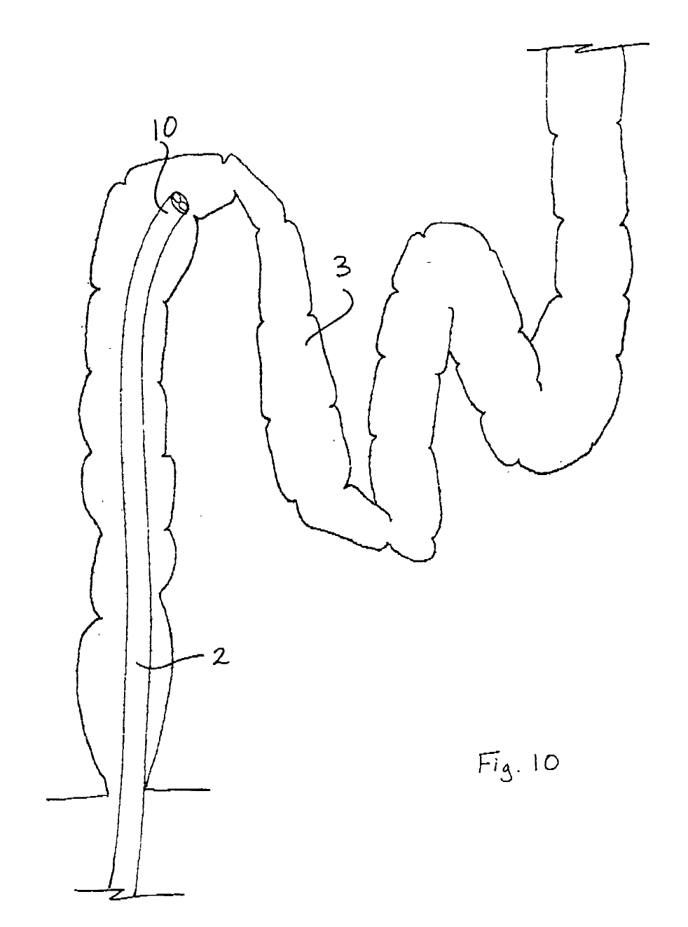

Inflation of the balloon 21 expands the balloon 21 outwardly into an anchored engagement with the interior wall of the colon 3, as illustrated in FIG. 12.

The longitudinal length of the balloon 21 may be selected to achieve a desired anchoring between the colon 3 and the balloon 21.

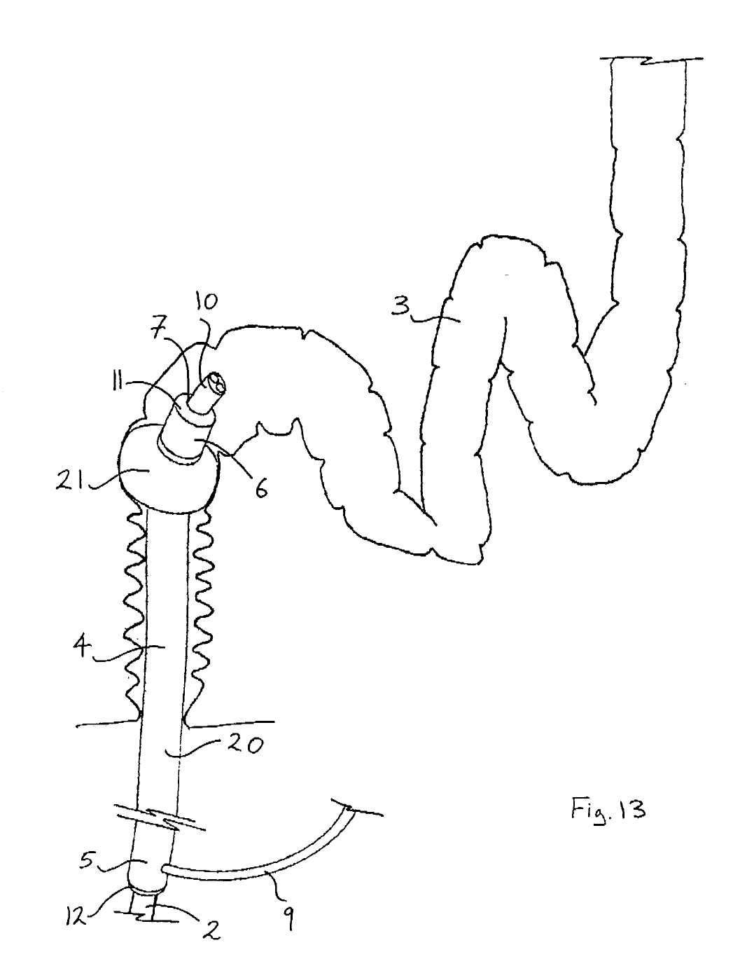

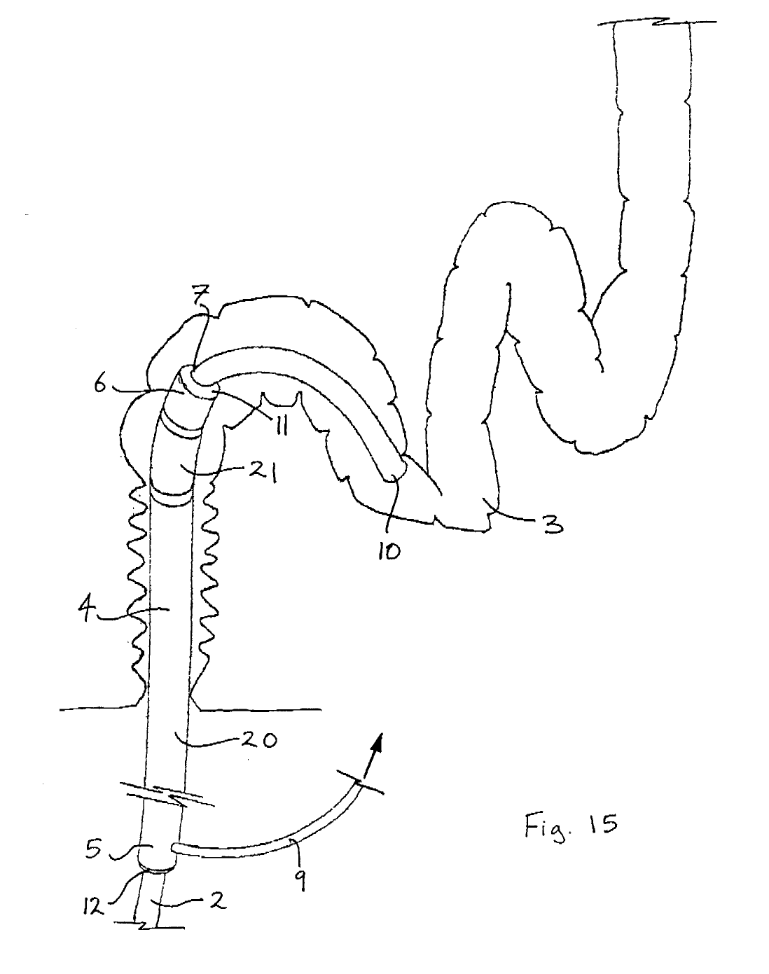

FIGS. 10 to 18 illustrate the device 20 being used with the colonoscope 2 to advance the colonoscope 2 through the colon 3, and in this case also to straighten the sigmoid colon, in a manner similar to that described previously with reference to FIGS. 1 to 9. In the case of FIGS. 10 to 18, the device 20 is anchored to the interior wall of the colon 3 by inflation of the balloon 21 to expand the balloon 21 outwardly into an anchored engagement with the interior wall of the colon 3 (FIGS. 12 and 17), and the device 20 releases the anchor of the interior wall of the colon 3 to the device 20 by deflating the balloon 21 (FIG. 15).

It will be appreciated that the anchor of the device of the invention may be provided by one or more arrays of suction apertures 8, as described previously with reference to FIGS. 1 to 9, or by one or more outwardly moveable members, such as the inflatable balloon 21, as described previously with reference to FIGS. 10 to 18, or by one or more arrays of suction apertures 8 in combination with one or more outwardly moveable members.

Referring now to FIGS. 19 and 20, there is illustrated another device 101 according to the invention, which is similar to the device 20 of FIGS. 10 to 18, and similar elements in FIGS. 19 and 20 are assigned the same reference numerals.

In this case, the anchor comprises an absorber, the absorber engaging with the interior wall of the colon 3 upon inflation of the balloon 21 into the anchored engagement with the interior wall of the colon 3.

It is believed that the absorber absorbs some of the fluids and/or faeces from the colon 3 in the region of anchoring of the interior wall of the colon 3 to the device 101, and in this manner a more secure anchoring engagement is achieved.

The absorber is provided, in this case, by three hoops 100 of a porous material, such as a sponge or a foam, mounted directly to the balloon 21, such as by an adhesive. The hoops 100 extend around the balloon 21, as illustrated in FIG. 20.

The device 101 further comprises a movable sheath 102 which may be positioned around the absorption hoops 100 to separate the absorption hoops 100 from the interior wall of the colon 3 during insertion and removal of the device 101 from the colon 3. In this way, the device 101 can be inserted into the colon 3 and removed from the colon 3 with less frictional resistance. Prior to balloon inflation, the sheath 102 is retracted to reveal the absorption hoops 100.

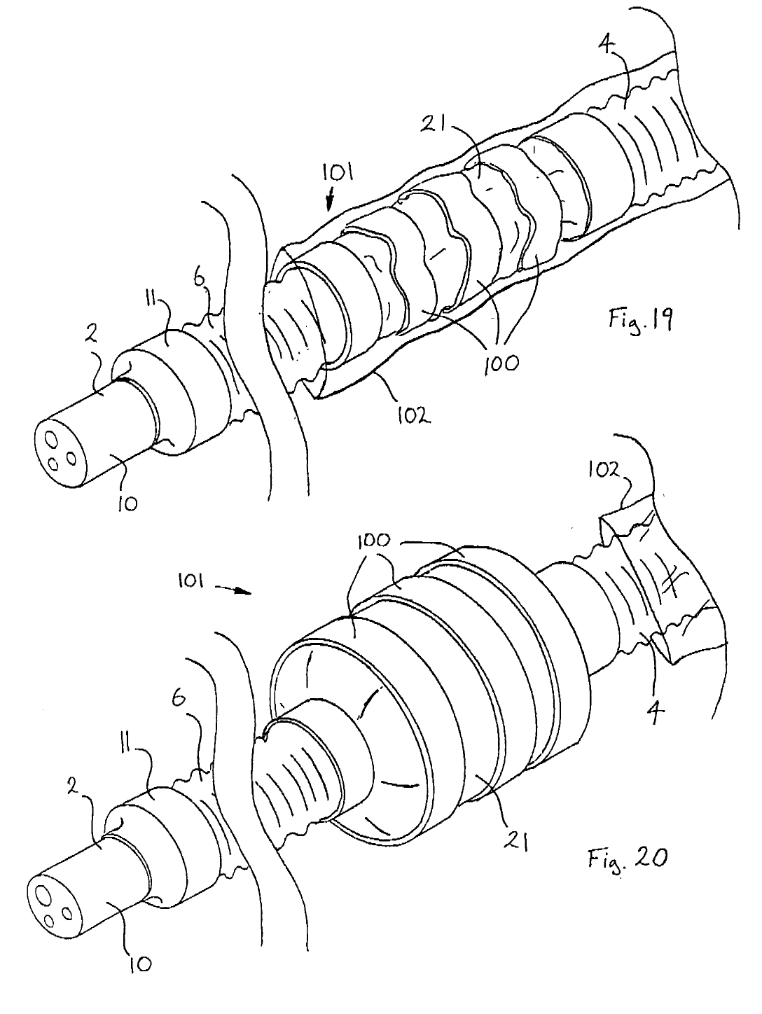

It will be appreciated that the absorber is suitable, in use, to enhance the anchoring engagement between the interior wall of the colon 3 with a cylindrical balloon 21, as illustrated in FIGS. 19 and 20, or a spherical balloon 112, as illustrated in FIGS. 21 and 22, or any other type of balloon.

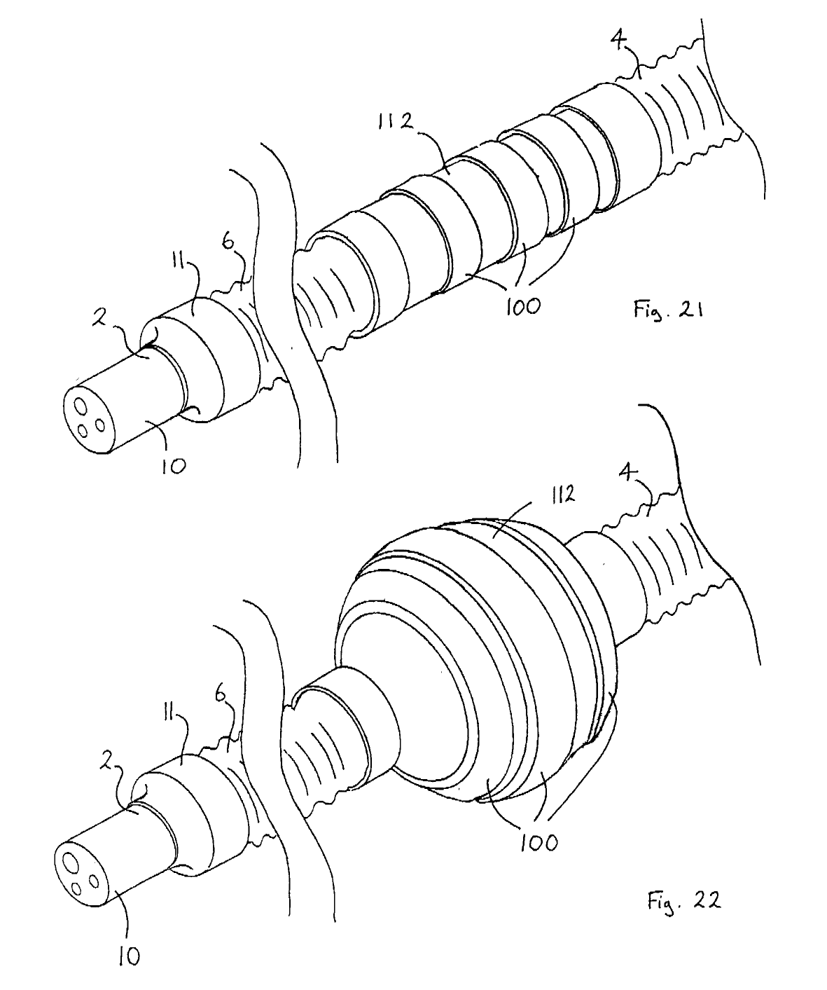

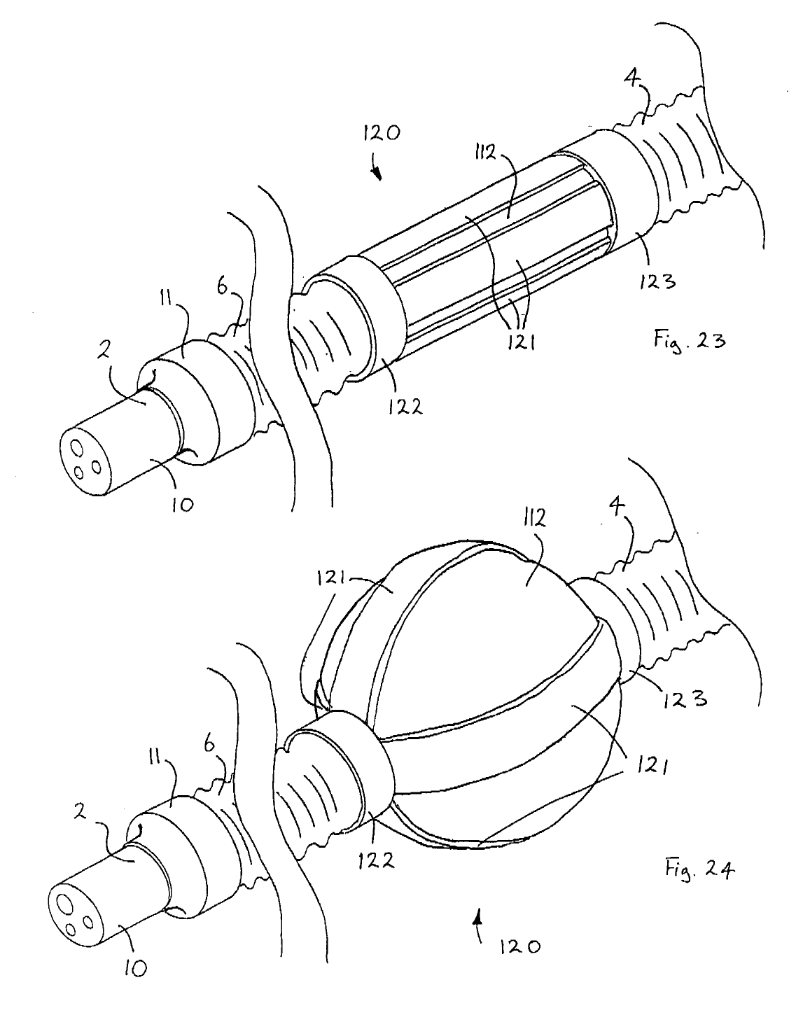

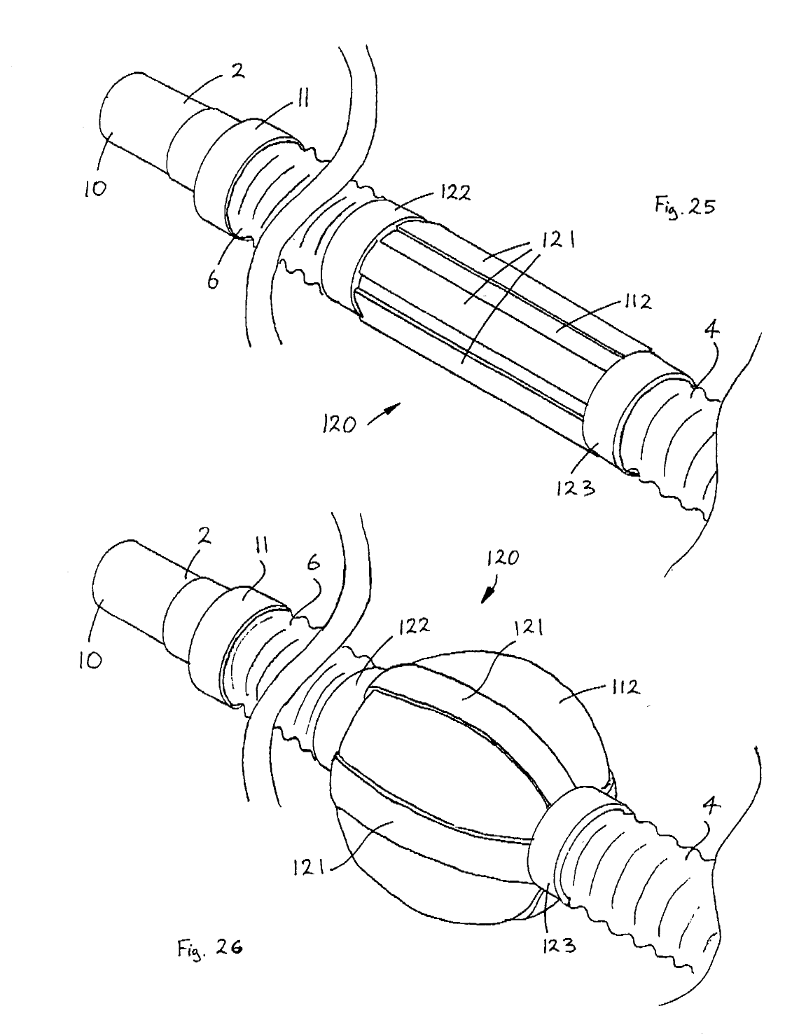

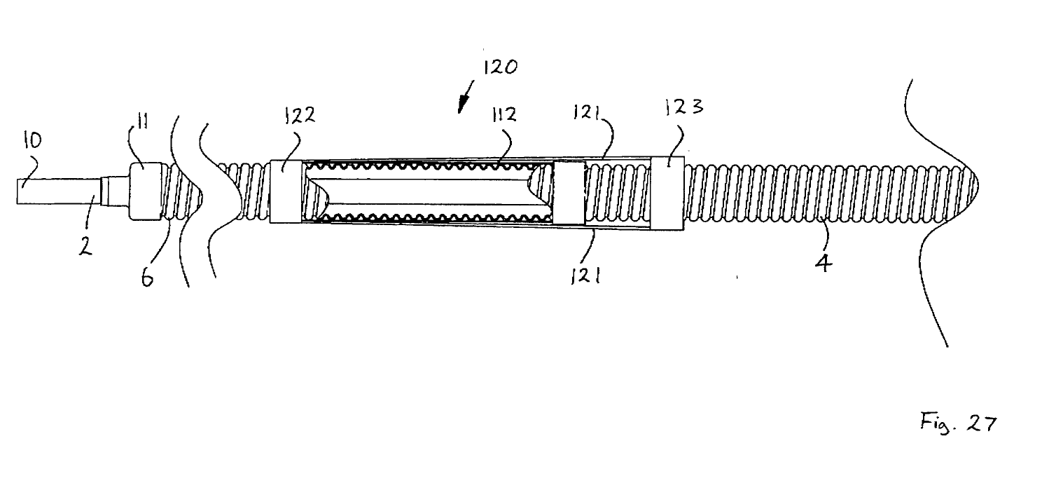

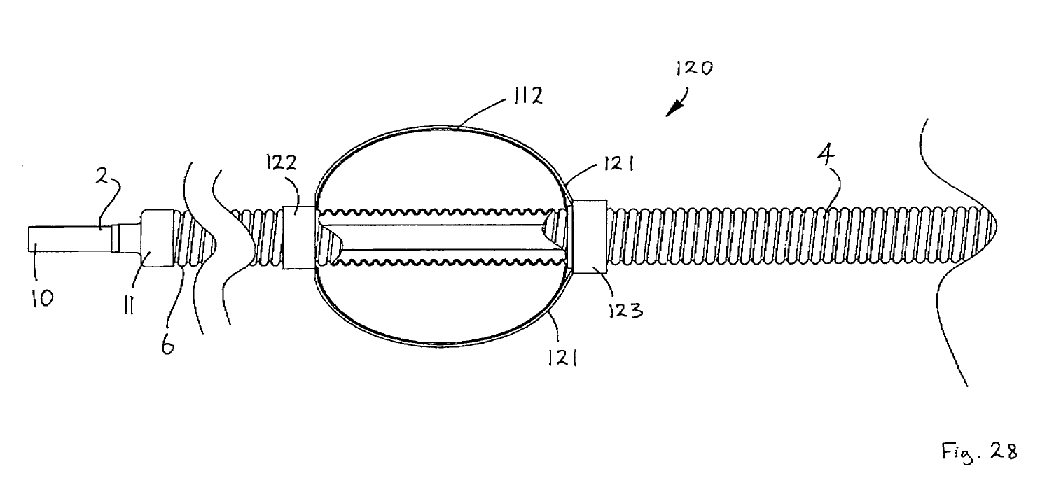

FIGS. 23 to 28 illustrate another device 120 according to the invention, which is similar to the device 101 of FIGS. 19 and 20, and similar elements in FIGS. 23 to 28 are assigned the same reference numerals.

In this case, the absorber is provided by a plurality of strips 121 of porous material which extend longitudinally along the spherical balloon 112. The strips 121 are mounted to the body 4 by means of a distal collar 122 and a proximal collar 123. The distal collar 122 is fixed to the body 4, and the proximal collar 123 is slidable distally over the body 4 as the balloon 112 is inflated from the uninflated configuration (FIGS. 23, 25, 27) to the inflated configuration (FIGS. 24, 26, 28). Upon deflation of the balloon 112, the proximal collar 123 slides proximally back to the uninflated configuration.

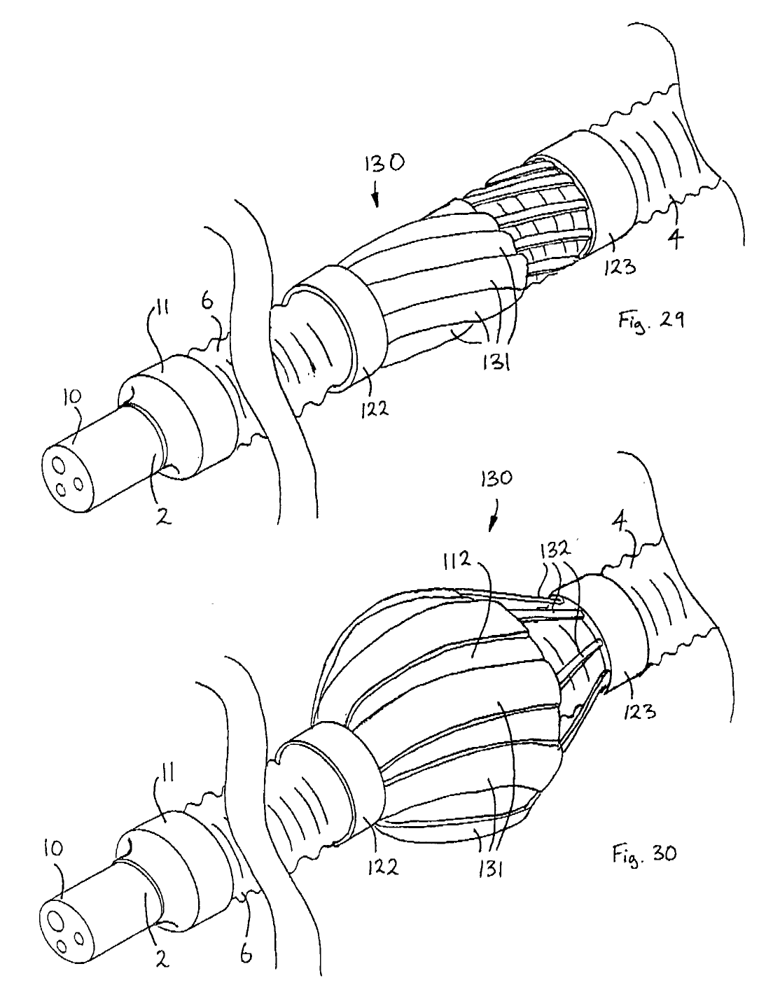

Referring to FIGS. 29 and 30, there is illustrated another device 130 according to the invention, which is similar to the device 120 of FIGS. 23 to 28, and similar elements in FIGS. 29 and 30 are assigned the same reference numerals.

The absorber is provided, in this case, by a plurality of strips 131 of porous material which extend in a spiral along the spherical balloon 112. The strips 131 are connected to the proximal ring 123 by means of a plurality of resilient bands 132. The proximal ring 123 is fixed to the body 4, in this case, such that the bands 132 bias the strips 131 inwardly to a low-profile configuration (FIG. 29) for ease of insertion and removal of the device 130 from the colon 3.

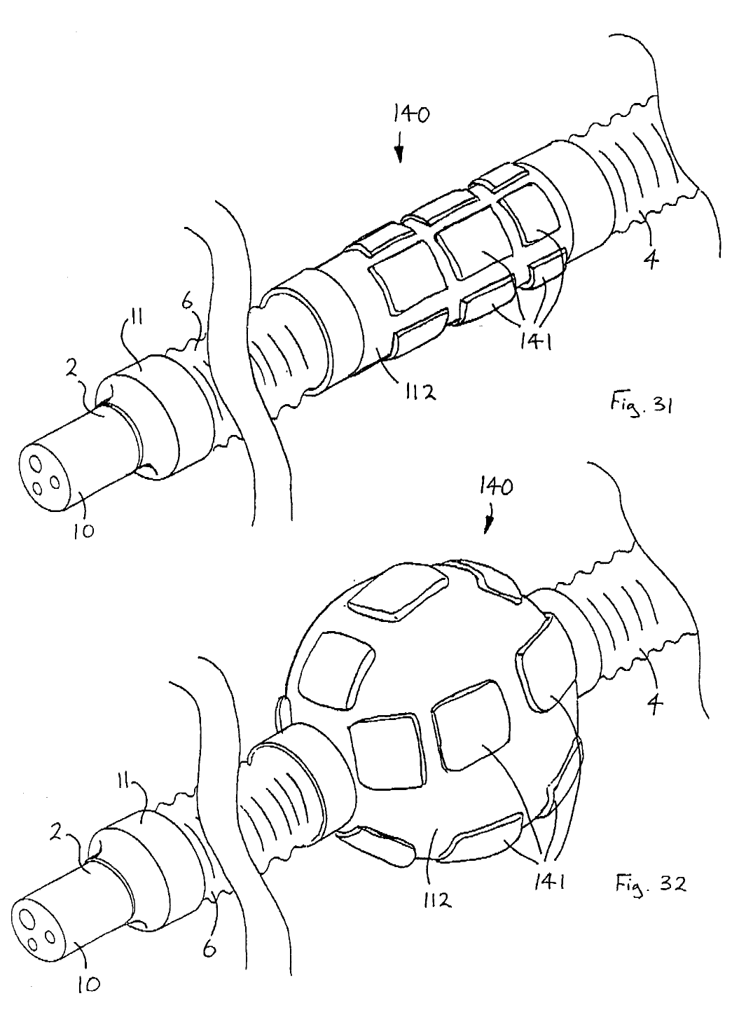

FIGS. 31 to 37 illustrate further devices 140, 150, 160 according to the invention, which are similar to the device 101 of FIGS. 19 and 20, and similar elements in FIGS. 31 to 37 are assigned the same reference numerals.

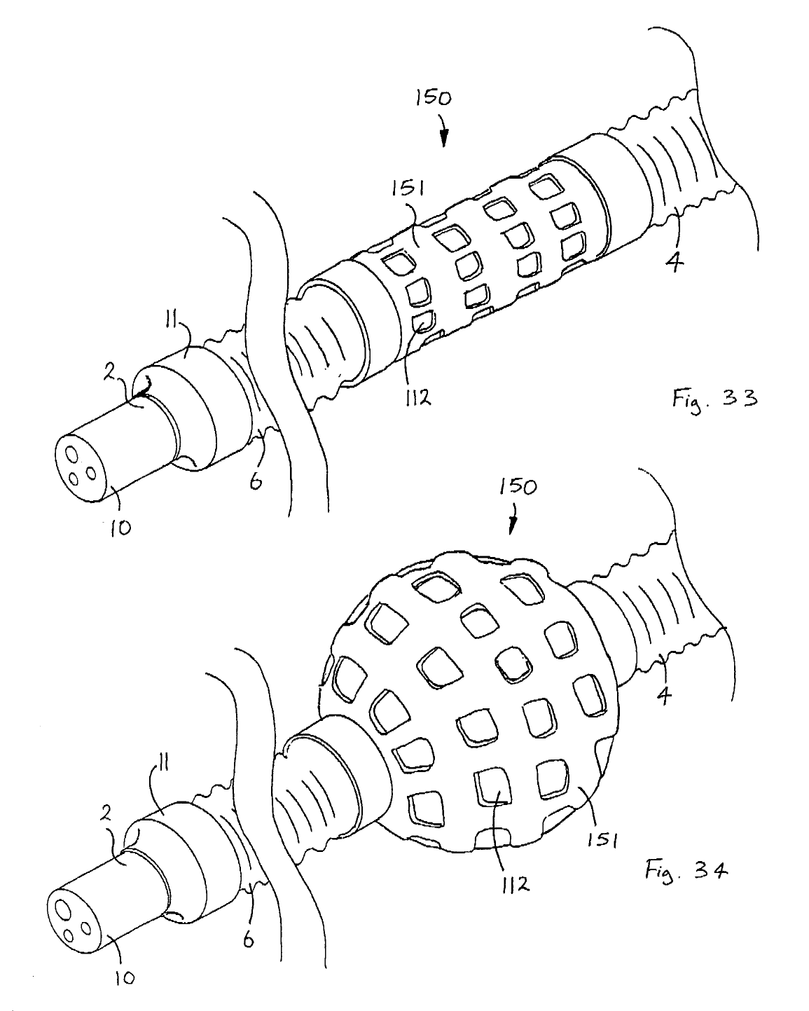

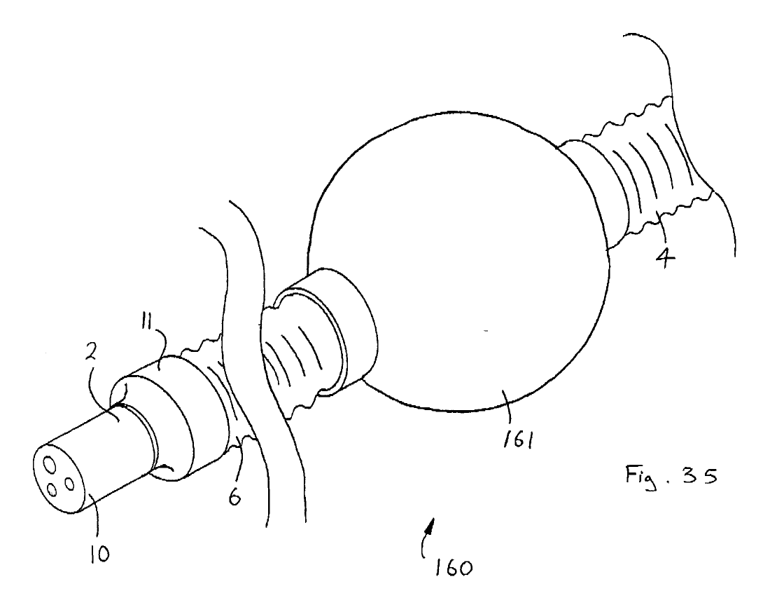

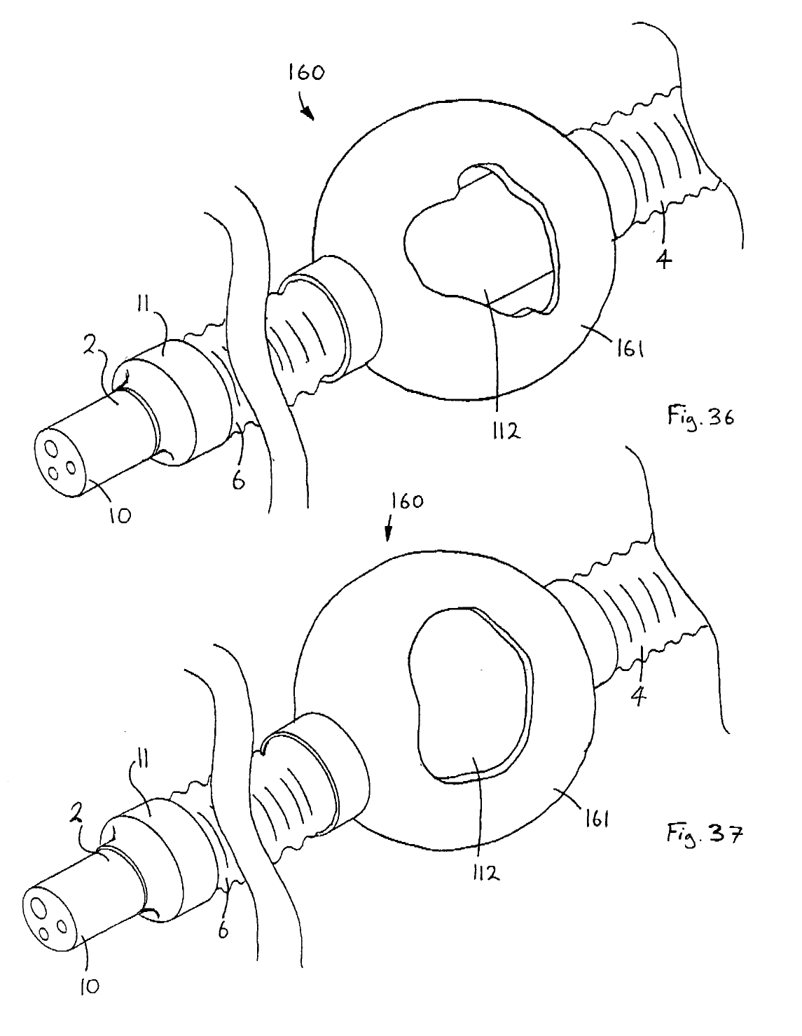

In the case of the device 140 of FIGS. 31 and 32, the absorber is provided by a plurality of pads 141 or blocks of porous material adhesively fixed to the spherical balloon 112. In the device 150 of FIGS. 33 and 34, the absorber 151 is adhesively fixed around the spherical balloon 112 to surround the balloon 112. A plurality of openings are provided in the absorber 151.

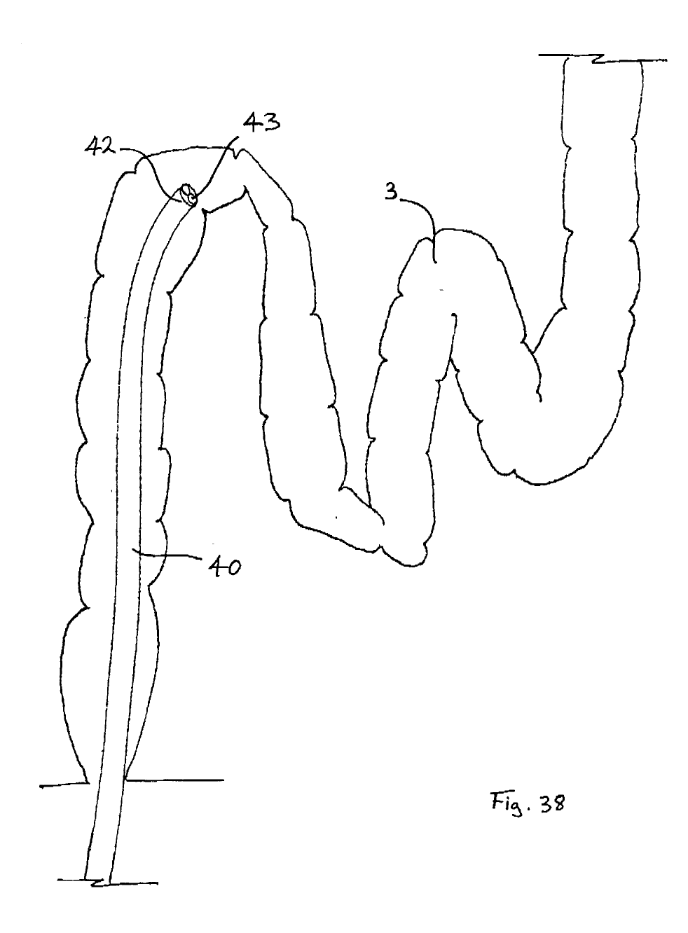

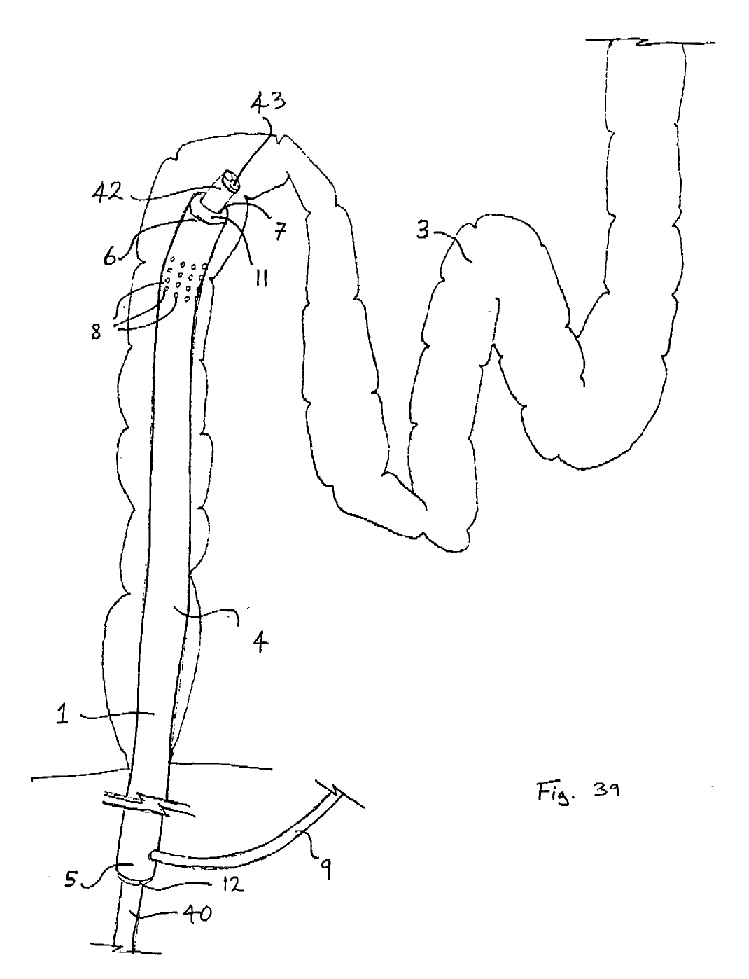

In FIGS. 35 to 37, the device 160 comprises a spherical absorber 161 which forms a continuous enclosure surrounding the balloon 112. As illustrated in FIGS. 36 and 37, the absorber 161 is biased to extend outwardly in both the inflated and uninflated configurations. However when the balloon 112 is deflated, the absorber 161 is relatively flexible and may be collapsed down around the deflated balloon 112 for a low-profile.

The absorber may be of any suitable porous material, preferably of open cell construction, for example a polymeric material, such as polyurethane.

It will be appreciated that an absorber, similar to that described previously with reference to FIGS. 19 to 37, may be used with the device 1 described previously with reference to FIGS. 1 to 9 to enhance the anchoring engagement of the interior wall of the colon 3 to the body 4.

Referring next to FIGS. 38 to 44, the device 1 of the invention, which was described previously with reference to FIGS. 1 to 9, is illustrated in use aiding advancement of a colonoscope 40 through the colon 3.

The colonoscope 40 defines a proximal end for location externally of the colon 3 and a distal end 42 for insertion into the colon 3.

The colonoscope 40 comprises an insufflation means at the distal end 42 of the colonoscope 40 for insufflating the colon 3 distally of the colonoscope 40 to clear a passage for advancement of the colonoscope 40 through the colon 3. In this case, the insufflation means comprises at least one insufflation aperture 43 in the colonoscope 40 at the distal end 42 of the colonoscope 40, the aperture 43 being in fluid communication with at least one insufflation lumen. The insufflation lumen extends proximally through the colonoscope 40 to the proximal end of the colonoscope 40, where the lumen is connected in fluid communication with an insufflation fluid source.

In use, the distal end 42 of the colonoscope 40 is inserted into the colon 3, and the colonoscope 40 is advanced through the colon 3 until the distal end 42 of the colonoscope 40 encounters an obstruction to further advancement of the colonoscope 40 through the colon 41, such as a tight bend in the sigmoid colon, as illustrated in FIG. 38. It is not possible to easily advance the colonoscope 40 any further distally through this tight bend in the sigmoid colon.

The distal end 6 of the body 4 is inserted into the colon 3, and the body 4 is advanced over the colonoscope 40 until the distal end 6 of the body 4 is immediately proximally of the distal end 42 of the colonoscope 40 (FIG. 39)

The interior wall of the colon 3 is anchored to the device 1 by applying suction at the suction apertures 8 to draw the interior wall of the colon 3 into an anchored engagement with the body 4. By holding the device 1 in position, the anchored colon 3 will also be held in position. The colonoscope 40 may then be advanced more easily through the colon 3.

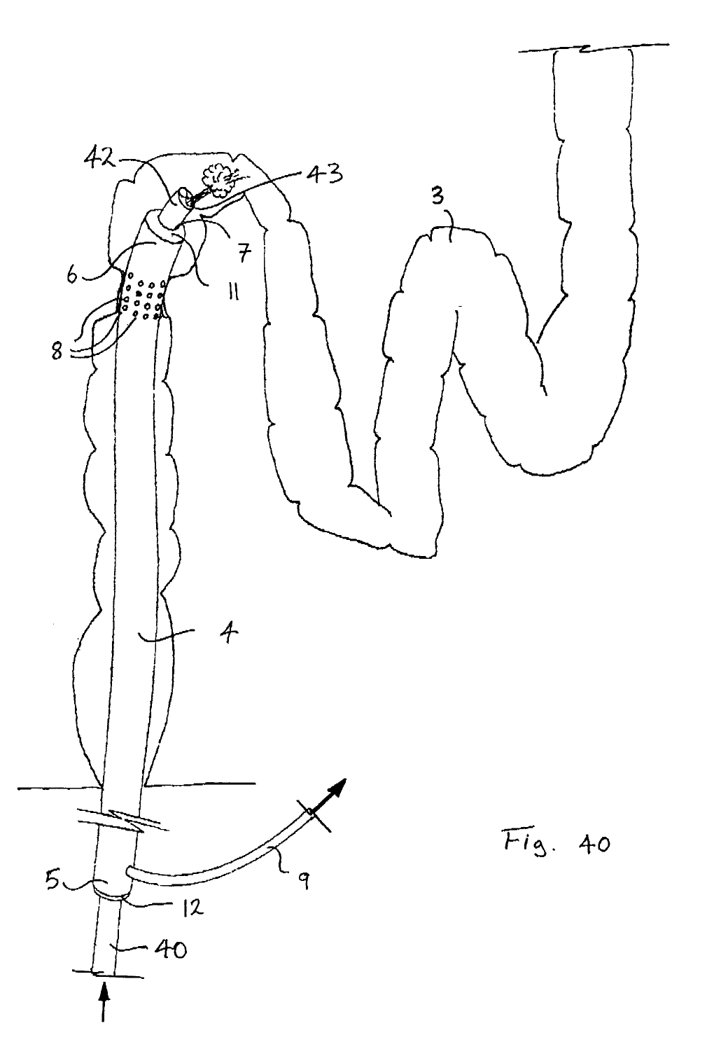

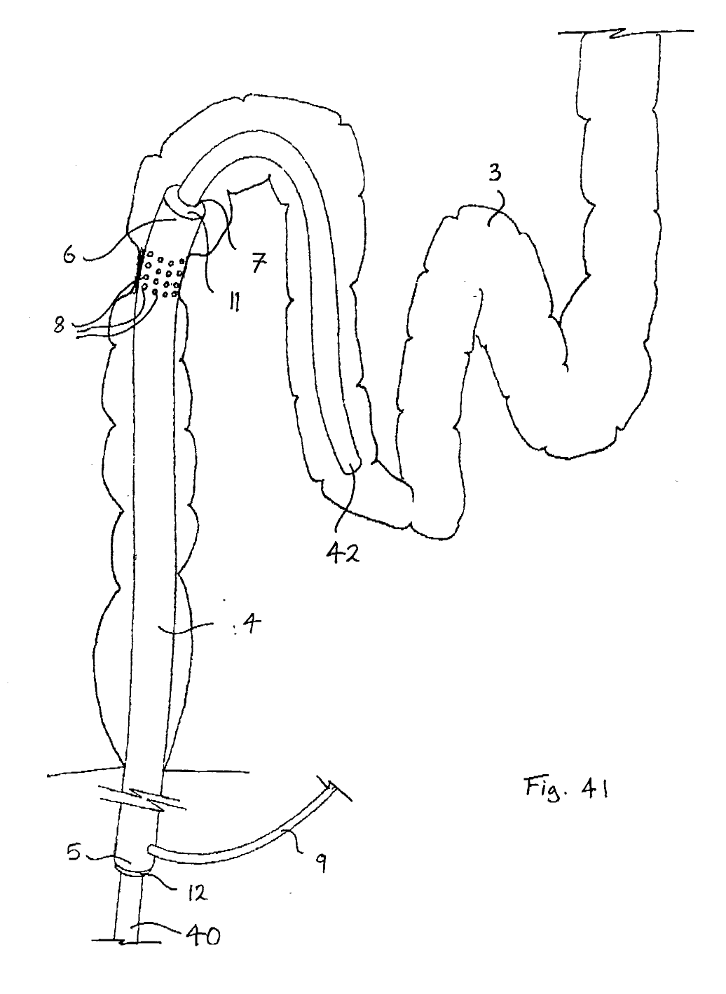

In this case, the method also includes the step of passing insufflation fluid from the insufflation fluid source through the insufflation lumen in the colonoscope 40 and out of the insufflation aperture 43 to insufflate the colon 3 distally of the colonoscope 40 (FIG. 40). The insufflation action helps to open up the bend in the colon 3 and in this way clears a passage for further advancement of the colonoscope 40 through the colon 3 (FIG. 41).

By anchoring the interior wall of the colon 3 to the body 4, this also creates a fluid-tight seal between the interior wall of the colon 3 and the body 4.

By creating the seal proximally of the insufflation aperture 43, this ensures that no insufflation fluid can leak proximally over the colonoscope 40. In this manner, the full effect of the insufflation is directed distally to effectively clear a passage for advancement of the colonoscope 40 through the colon 3, as illustrated in FIG. 41.

The seals 11, 12 also prevent insufflation fluid from leaking proximally between the colonoscope 40 and the body 4.

Insufflation of the colon 41 may be stopped before further advancement of the colonoscope 40, alternatively the insufflation may continue during the advancement of the colonoscope 40 to ensure an unobstructed passage through the colon 3.

During this further advancement of the colonoscope 40 through the now cleared colon 3, the anchor between the body 4 and the interior wall of the colon 3 is maintained, and in this way the seal between the body 44 and the colon 3 is also maintained.

After the colonoscope 40 has been advanced further, the anchoring of the body 4 to the interior wall of the colon 3 may be released by releasing the suction.

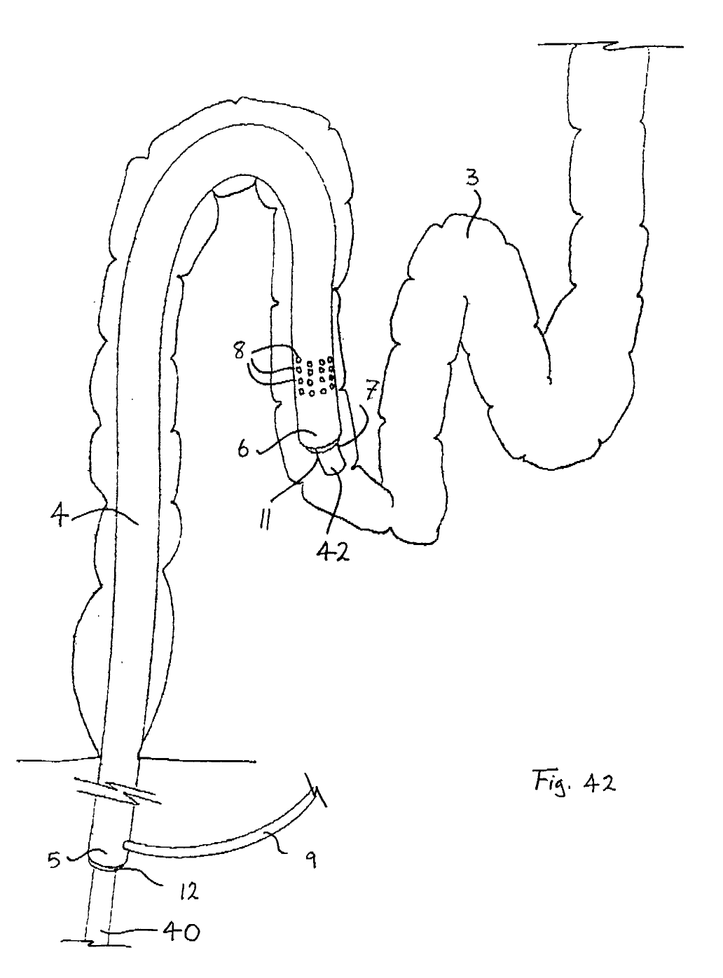

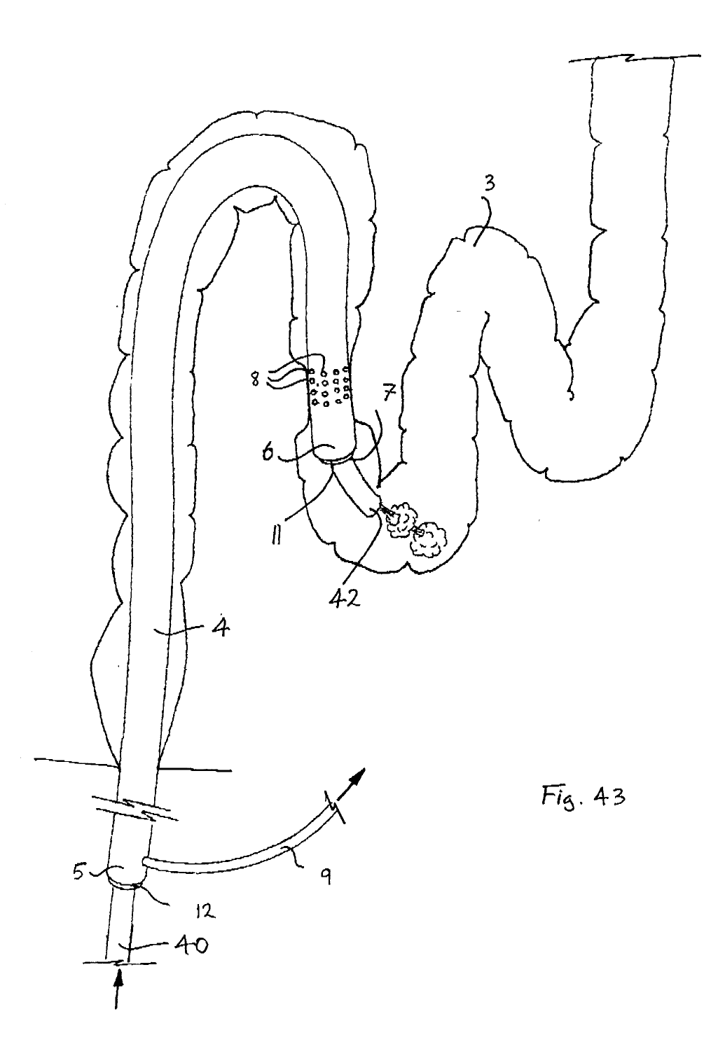

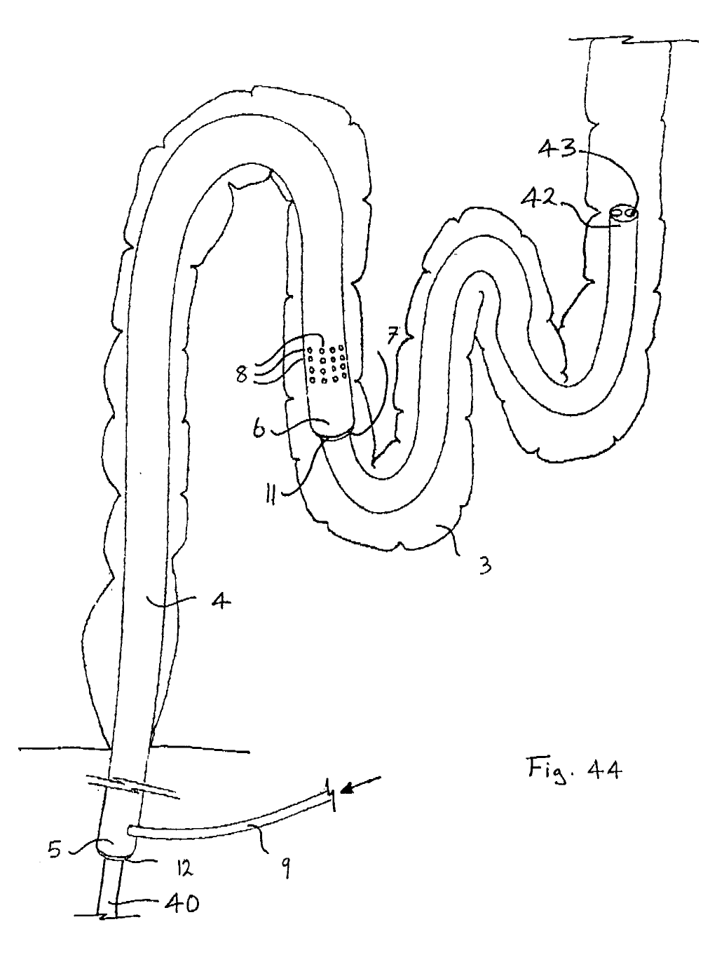

The colonoscope 40 is advanced through the colon 3 until another obstruction to further advancement of the colonoscope 40 is encountered, such as a tight bend in the sigmoid colon, as illustrated in FIG. 41. The body 4 is then advanced until the distal end 6 of the body 4 is immediately proximally of the distal end 42 of the colonoscope 40 (FIG. 42). The interior wall of the colon 3 is anchored to the body 4 by applying suction at the suction apertures 8, and the colon 3 is insufflated distally of the colonoscope 40 to open up the bend and clear a passage for further advancement of the colonoscope 40 through the colon 3 (FIG. 43), in a manner similar to that described previously with reference to FIG. 40.

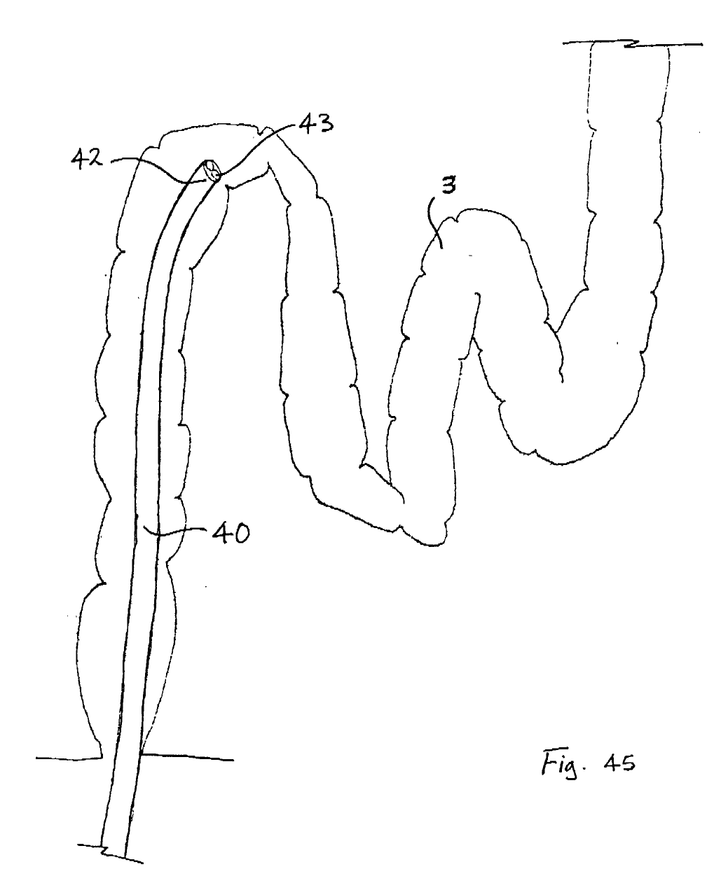

The colonoscope 40 is then advanced further distally through the colon 3 unobstructed, after which the anchoring of the body 4 to the interior wall of the colon 3 may be released by releasing the suction (FIG. 44).

This procedure may be repeated to clear a passage for advancement of the colonoscope 40 through the colon 3 each time an obstruction is encountered. As illustrated in FIGS. 38 to 44, as the procedure is repeated through the colon 3, the point of anchoring of the body 4 to the interior wall of the colon 3 progresses successively distally.

When the colonoscope 40 has been advanced through a colon 3 until the distal end 42 of the colonoscope 40 is at the proximal end of the descending colon (FIG. 44), the sigmoid colon may be straightened by manipulating and/or torquing the colonoscope 40 in a manner known to those skilled in the art.

The device 1 is then advanced over the colonoscope 40 until the distal end 6 of the body 3 is immediately proximally of the distal end 42 of the colonoscope 40 which is in the descending colon.

The device 1 then may act as a splint to maintain the floppy sigmoid colon in the straightened configuration, and thus the colonoscope 40 may be advanced further distally through the descending colon, into the transverse colon, and into the ascending colon without causing pain and/or discomfort to the patient.

It is believed that anchoring the interior wall of the colon 3 by means of suction results in a secure anchor of the colon wall to the device 1 without causing discomfort to the patient.

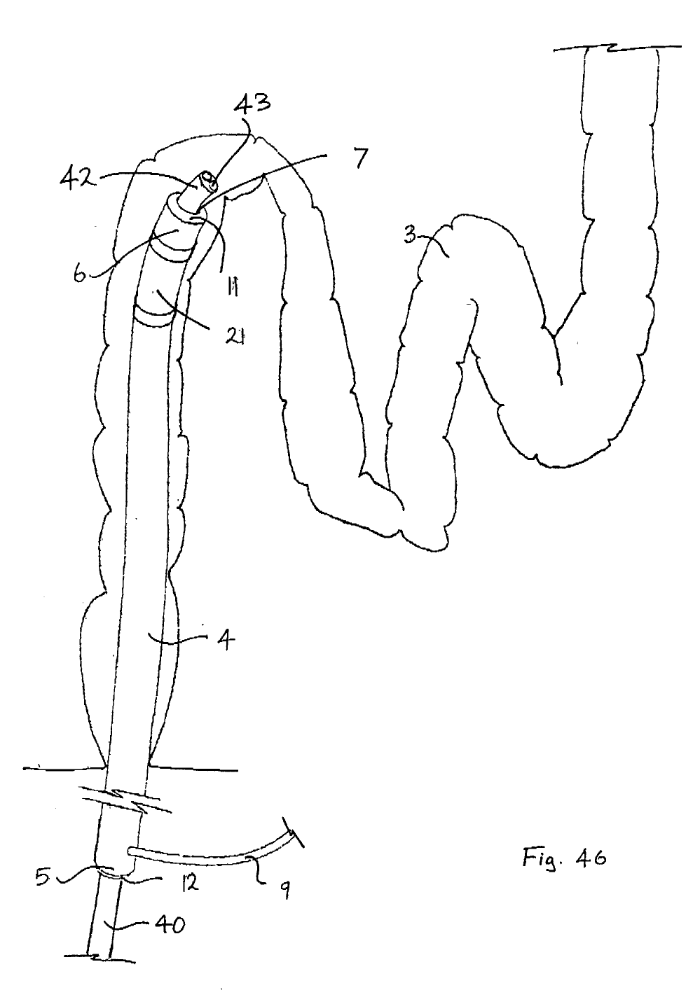

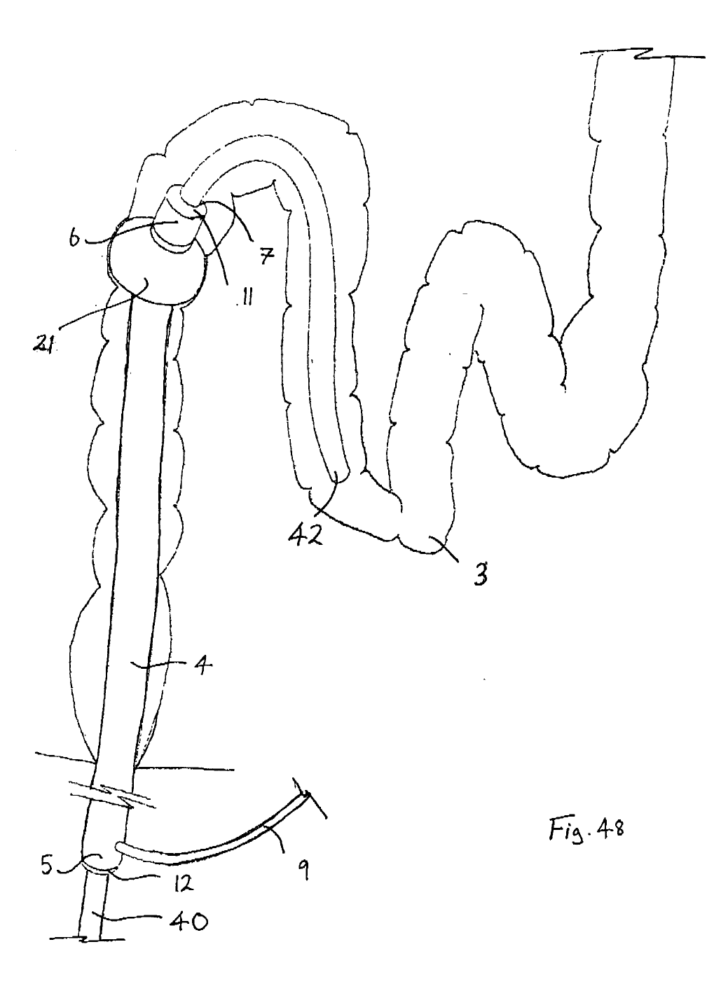

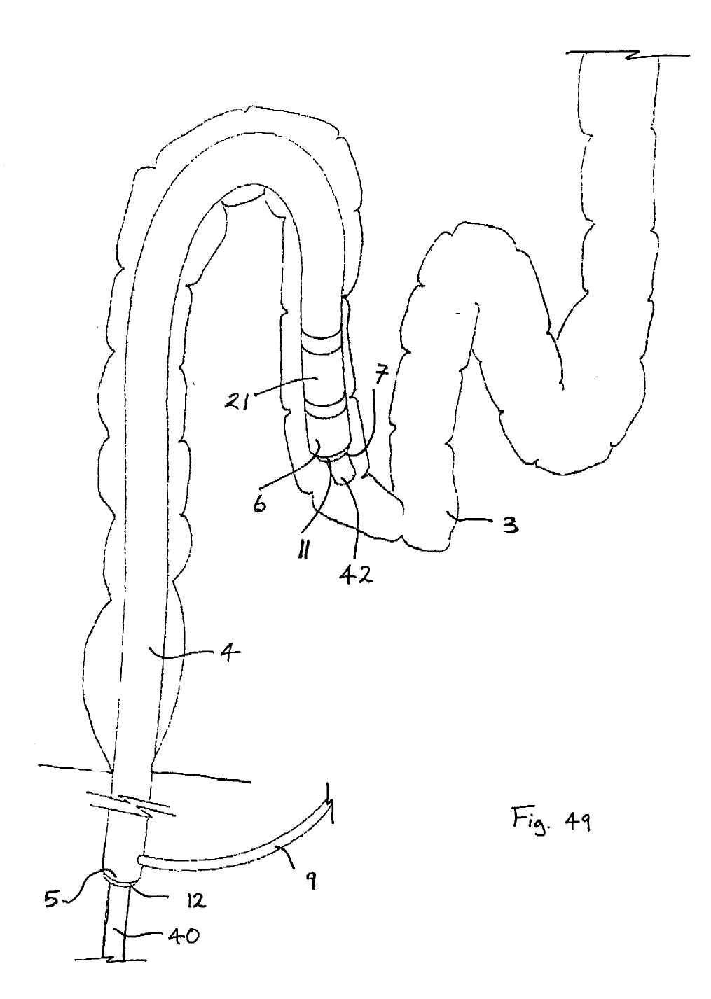

Referring to FIGS. 45 to 49, the device 20 of the invention, which was described previously with reference to FIGS. 10 to 18, is illustrated in use aiding advancement of the colonoscope 40 through the colon 3 in a manner similar to that described with reference to FIGS. 38 to 44.

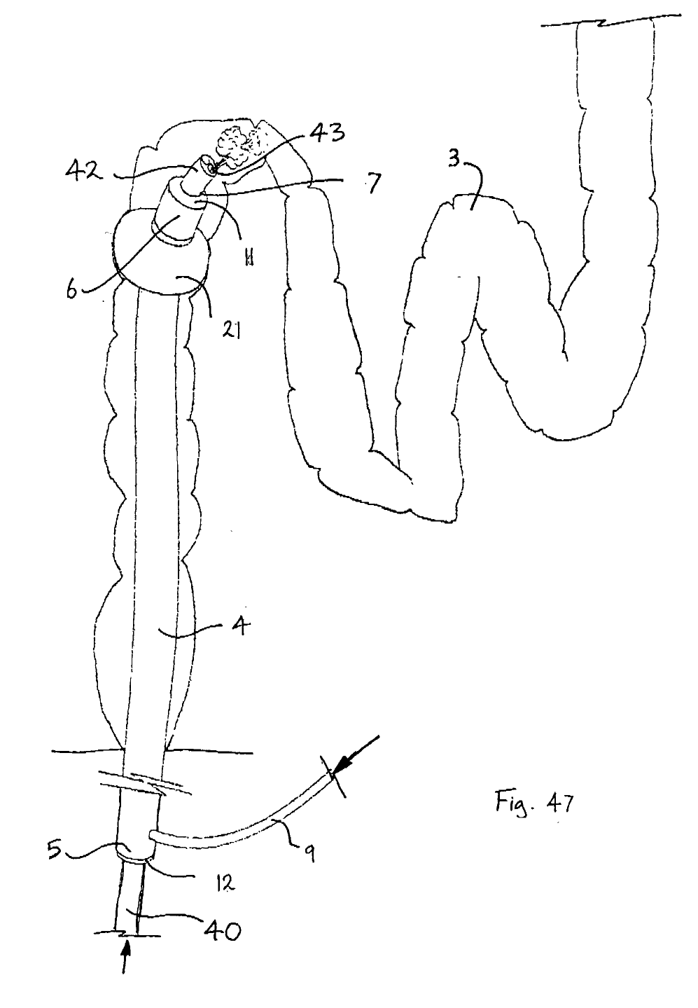

In use, the interior wall of the colon 3 is anchored to the device 20 by passing inflation fluid from the inflation fluid source through the tubing 9 into the inflation lumen, through the inflation lumen and into the balloon 21 to inflate the balloon 21 outwardly into an anchored engagement with the interior wall of the colon 3 (FIG. 47).

The anchoring of the body 4 to the interior wall of the colon 3 is subsequently released by deflating the balloon 21 to move the balloon 21 inwardly.

It is believed that the use of an absorber, as described previously with reference to FIGS. 19 to 37, also enhances the sealing of the balloon 21 to the interior wall of the colon 3.

The procedure for advancing a colonoscope through a colon has been described in detail and illustrated in FIGS. 3(a) to 49 in relation to advancement of the colonoscope through the sigmoid colon. It will of course be appreciated that the device of the invention is suitable for aiding advancement of a colonoscope through any part of the colon.

It will further be appreciated that the straightening procedure, as described previously with reference to FIGS. 3(a) to 18, may be used in combination with the insufflation procedure, as described previously with reference to FIGS. 38 to 49.

The invention is not limited to the embodiments hereinbefore described, with reference to the accompanying drawings, which may be varied in construction and detail.

1. A device to aid advancement of a colonoscope through a colon, the device comprising:
an elongate body extending between a proximal end for location externally of a colon and a distal end for insertion into a colon;
the body defining a colonoscope lumen therethrough, and
an anchor to releasably anchor the interior wall of a colon to the device.

2. A device as claimed in claim 1 wherein the anchor comprises at least one suction aperture in the body to draw the interior wall of a colon into an anchored engagement with the body by suction.

3. A device as claimed in claim 2 wherein the anchor comprises a plurality of suction apertures.

4. A device as claimed in claim 3 wherein the apertures are evenly spaced around the body.

5. A device as claimed in claim 3 wherein the apertures are evenly spaced along the body.

6. A device as claimed in claim 2 wherein the anchor comprises at least one suction lumen in fluid communication with the suction aperture.

7. A device as claimed in claim 6 wherein the suction lumen extends proximally from the suction aperture to the proximal end of the body.

8. A device as claimed in claim 6 wherein the suction lumen is provided by the colonoscope lumen.

9. A device as claimed in claim 2 wherein the anchor comprises a source of negative gauge pressure in fluid communication with the suction aperture.

10. A device as claimed in claim 1 wherein the anchor comprises at least one member which is moveable outwardly into an anchored engagement with the interior wall of a colon.

11. A device as claimed in claim 10 wherein the member is expandable into an anchored engagement with the interior wall of a colon.

12. A device as claimed in claim 11 wherein the member is inflatable into an anchored engagement with the interior wall of a colon.

13. A device as claimed in claim 12 wherein the inflatable member comprises a balloon.

14. A device as claimed in claim 12 wherein the anchor comprises at least one inflation lumen in fluid communication with the inflatable member.

15. A device as claimed in claim 14 wherein the inflation lumen extends proximally from the inflatable member to the proximal end of the body.

16. A device as claimed in claim 14 wherein the inflation lumen is provided by the colonoscope lumen.

17. A device as claimed in claim 12 wherein the anchor comprises an inflation fluid source in fluid communication with the inflatable member.

18. A device as claimed in claim 1 wherein the anchor comprises an absorber.

19. A device as claimed in claim 18 wherein the absorber is mounted to the elongate body.

20. A device as claimed in claim 19 wherein the absorber is biased inwardly.

21. A device as claimed in claim 19 wherein the absorber is biased outwardly.

22. A device as claimed in claim 18 wherein the absorber is mounted to or comprises the movable member.

23. A device as claimed in claim 18 wherein the absorber comprises at least one pad on the device.

24. A device as claimed in claim 18 wherein the absorber comprises at least one strip extending along the device.

25. A device as claimed in claim 24 wherein the strip extends substantially longitudinally.

26. A device as claimed in claim 24 wherein the strip extends substantially in a spiral or part thereof.

27. A device as claimed in claim 18 wherein the absorber comprises at least one hoop extending around the device.

28. A device as claimed in claim 18 wherein the absorber surrounds the anchor.

29. A device as claimed in claim 28 wherein the absorber has a plurality of openings therein.

30. A device as claimed in claim 28 wherein the absorber is a continuous enclosure around the anchor.

31. A device as claimed in claim 18 wherein the absorber is of a porous material.

32. A device as claimed in claim 31 wherein the porous material is of an open cell construction.

33. A device as claimed in claim 31 wherein the porous material is of a polymeric material.

34. A device as claimed in claim 18 wherein the device comprises a sleeve to separate the absorber from the interior wall of a colon during insertion and removal of the device.

35. A device as claimed in claim 34 wherein the sleeve comprises a movable sheath.

36. A device as claimed in claim 1 wherein the anchor is provided adjacent the distal end of the body.

37. A device as claimed in claim 1 wherein the body comprises at least one seal for sealing between the body and a colonoscope within the colonoscope lumen.

38. A device as claimed in claim 37 wherein the seal is a fluid-tight seal.

39. A device as claimed in claim 37 wherein the seal is provided at an end of the body.

40. A device as claimed in claim 39 wherein the body comprises a distal seal at the distal end of the body and a proximal seal at the proximal end of the body.

41. A colonoscopy apparatus, the apparatus comprising:
a device to aid advancement of a colonoscope through a colon, the device comprising:
an elongate body extending between a proximal end for location externally of a colon and a distal end for insertion into a colon;
the body defining a colonoscope lumen therethrough, and
an anchor to releasably anchor the interior wall of a colon to the device; and
a colonoscope defining a proximal end for location externally of a colon and a distal end for insertion into a colon.

42. An apparatus as claimed in claim 41 wherein the colonoscope comprises an insufflation means at the distal end of the colonoscope to insufflate a colon distally of the colonoscope to clear a passage for advancement of the colonoscope through the colon.

43. An apparatus as claimed in claim 42 wherein the insufflation means comprises at least one insufflation aperture in the colonoscope.

44. An apparatus as claimed in claim 43 wherein the insufflation means comprises at least one insufflation lumen in fluid communication with the insufflation aperture.

45. An apparatus as claimed in claim 44 wherein the insufflation lumen extends proximally from the insufflation aperture to the proximal end of the colonoscope.

46. An apparatus as claimed in claim 43 wherein the insufflation means comprises an insufflation fluid source in fluid communication with the insufflation aperture.

47. An apparatus as claimed in claim 42 wherein the apparatus comprises a sealing means to create a fluid-tight seal to the interior wall of a colon proximally of the distal end of the colonoscope during insufflation.

48. An apparatus as claimed in claim 47 wherein the sealing means is provided by the anchor.

49. A method of advancing a colonoscope through a colon, the method comprising the steps of:
providing a colonoscope, the colonoscope defining a proximal end and a distal end;
providing an anchoring device, the anchoring device defining a proximal end and a distal end;
inserting the distal end of the colonoscope into a colon;
inserting the distal end of the anchoring device into the colon;
anchoring the interior wall of the colon to the anchoring device;
advancing the colonoscope through at least part of the colon; and
releasing the anchor.

50. A method as claimed in claim 49 wherein the method comprises the step of moving the anchoring device proximally to at least partially straighten the colon after anchoring the interior wall of the colon to the anchoring device.

51. A method as claimed in claim 50 wherein the method comprises the step of moving the colonoscope proximally during the step of moving the anchoring device proximally.

52. A method as claimed in claim 50 wherein the method comprises the step of maintaining the position of the colonoscope fixed during the step of moving the anchoring device proximally.

53. A method as claimed in claim 49 wherein the method comprises the step of insufflating the colon distally of the colonoscope to clear a passage for advancement of the colonoscope through the colon.

54. A method as claimed in claim 53 wherein the method comprises the step of creating a fluid-tight seal to the interior wall of the colon proximally of the distal end of the colonoscope before insufflating the colon.

55. A method as claimed in claim 54 wherein the seal to the interior wall of the colon is created by the step of anchoring the interior wall of the colon to the anchoring device.

56. A method as claimed in claim 49 wherein the interior wall of the colon is anchored to the anchoring device by applying suction to draw the interior wall of the colon into an anchored engagement with the anchoring device.

57. A method as claimed in claim 56 wherein the anchor is released by releasing the suction.

58. A method as claimed in claim 49 wherein the interior wall of the colon is anchored to the anchoring device by moving at least part of the anchoring device outwardly into an anchored engagement with the interior wall of the colon.

59. A method as claimed in claim 58 wherein the part of the anchoring device is moved outwardly by inflation.

60. A method as claimed in claim 58 wherein the anchor is released by moving the part of the anchoring device inwardly.

61. A method as claimed in claim 49 wherein the method comprises the step of absorbing fluid from the colon using the anchoring device.

62. A method as claimed in claim 49 wherein the method comprises the step of advancing the colonoscope through the colon until the distal end of the colonoscope encounters an obstruction to further advancement of the colonoscope through the colon before anchoring the interior wall of the colon to the anchoring device.

63. A method as claimed in claim 62 wherein the colonoscope is advanced through the colon until the distal end of the colonoscope is at the proximal end of the sigmoid colon before anchoring the interior wall of the colon to the anchoring device.

64. A method as claimed in claim 49 wherein at least some of the steps are repeated at least once to advance the colonoscope through the colon in an incremental manner.

65. A method as claimed in claim 64 wherein the location of anchoring of the interior wall of the colon to the anchoring device progresses successively distally as the steps are repeated.

66. A method as claimed in claim 49 wherein the anchoring device is passed over the colonoscope.

67. A method as claimed in claim 66 wherein the anchoring device is passed over the colonoscope in a sealed manner.

68. A method of advancing a colonoscope through a sigmoid colon the method comprising the steps of:
providing a colonoscope, the colonoscope defining a proximal end and a distal end;
providing an anchoring device, the anchoring device defining a proximal end and a distal end;
inserting the distal end of the colonoscope into a sigmoid colon;
inserting the distal end of the anchoring device into the sigmoid colon;
anchoring the interior wall of the sigmoid colon to the anchoring device;
advancing the colonoscope through at least part of the sigmoid colon; and
releasing the anchor.

69. A method of straightening a sigmoid colon as claimed in claim 68 wherein the method comprises the step of moving the anchoring device proximally to at least partially straighten the sigmoid colon after anchoring the interior wall of the sigmoid colon to the anchoring device.

70. A method as claimed in claim 69 wherein the method comprises the steps of:
advancing the anchoring device until the distal end of the anchoring device is at the proximal end of the descending colon to splint the straightened sigmoid colon; and
advancing the colonoscope into the descending colon.
